SEQUENCE LISTING (39 sequences)

SEQ ID NO: 1 - RNA, 23 nt, synthetic construct
  misc_feature 1..23: note = gRNA
  source 1..23: mol_type = other RNA
gctgattttgttataaacaaagg

SEQ ID NO: 2 - RNA, 23 nt, synthetic construct
  misc_feature 1..23: note = gRNA
  source 1..23: mol_type = other RNA
ggaagtactcgagagagtaaagg

SEQ ID NO: 3 - RNA, 23 nt, synthetic construct
  misc_feature 1..23: note = gRNA
  source 1..23: mol_type = other RNA
ggatcactcattatcaacattgg

SEQ ID NO: 4 - RNA, 23 nt, synthetic construct
  misc_feature 1..23: note = gRNA
  source 1..23: mol_type = other RNA
gcattacaaataatgagcaatgg

SEQ ID NO: 5 - DNA, 31 bp, synthetic construct
  misc_feature 1..31: note = Single strand DNA oligonucleotide
  source 1..31: mol_type = other DNA
ccacactagtaacatggcaccaacaaatggc

SEQ ID NO: 6 - DNA, 36 bp, synthetic construct
  misc_feature 1..36: note = Single strand DNA oligonucleotide
  source 1..36: mol_type = other DNA
gaaaccagatcgttggtcaaaagtcacctatctttg

SEQ ID NO: 7 - DNA, 27 bp, synthetic construct
  misc_feature 1..27: note = Single strand DNA oligonucleotide
  source 1..27: mol_type = other DNA
atggcaccaacaaatggctctaccctc

SEQ ID NO: 8 - DNA, 29 bp, synthetic construct
  misc_feature 1..29: note = Single strand DNA oligonucleotide
  source 1..29: mol_type = other DNA
gcttttcctgtggcttttcccgtgaatgt

SEQ ID NO: 9 - DNA, 32 bp, synthetic construct
  misc_feature 1..32: note = Single strand DNA oligonucleotide
  source 1..32: mol_type = other DNA
gtacagtacacaatcttaggtatcatgtttgg

SEQ ID NO: 10 - DNA, 32 bp, synthetic construct
  misc_feature 1..32: note = Single strand DNA oligonucleotide
  source 1..32: mol_type = other DNA
taggctgagaaagtctttccactccaaagcct

SEQ ID NO: 11 - DNA, 25 bp, synthetic construct
  misc_feature 1..25: note = Single strand DNA oligonucleotide
  source 1..25: mol_type = other DNA
cttcttgtgatactaagtccaagca

SEQ ID NO: 12 - DNA, 22 bp, synthetic construct
  misc_feature 1..22: note = Single strand DNA oligonucleotide
  source 1..22: mol_type = other DNA
caggcttttgagtcccatttcg

SEQ ID NO: 13 - DNA, 25 bp, synthetic construct
  misc_feature 1..25: note = Single strand DNA oligonucleotide
  source 1..25: mol_type = other DNA
cttggaagaaagattcagtgagaag

SEQ ID NO: 14 - DNA, 20 bp, synthetic construct
  misc_feature 1..20: note = Single strand DNA oligonucleotide
  source 1..20: mol_type = other DNA
gacttcaggactctcccagt

SEQ ID NO: 15 - DNA, 21 bp, synthetic construct
  misc_feature 1..21: note = Single strand DNA oligonucleotide
  source 1..21: mol_type = other DNA
ccttcagagcaaagtccaaac

SEQ ID NO: 16 - DNA, 24 bp, synthetic construct
  misc_feature 1..24: note = Single strand DNA oligonucleotide
  source 1..24: mol_type = other DNA
gtatgacaaaatcagcaatatccc

SEQ ID NO: 17 - DNA, 20 bp, synthetic construct
  misc_feature 1..20: note = Single strand DNA oligonucleotide
  source 1..20: mol_type = other DNA
ttggaaactctttcgccact

SEQ ID NO: 18 - DNA, 20 bp, synthetic construct
  misc_feature 1..20: note = Single strand DNA oligonucleotide
  source 1..20: mol_type = other DNA
cagcaacaaaatacccgtct

SEQ ID NO: 19 - DNA, 322 bp, Citrus reticulata
  source 1..322: mol_type = genomic DNA
atggcaccaacaaatggctctaccctcgcaactgagtcctgggatattgctgattttgttattgtagtgatttaacaccccttaagactcaagatgctaagtcactgcccagaagactcaagttcttgatcaaacaagatgcagaagaaaccaagattagatatcaagttcccaactcaattccaataggctctagatctaatgattaatatcacagtgttttaacctaaacaagaaacaacacacaagaacagaatcttacgtggttcaattgtgcattacatgcacaatttacatccacgggaaaagccacaggaaaagc

SEQ ID NO: 20 - DNA, 323 bp, Citrus maxima
  source 1..323: mol_type = genomic DNA; note = Pumelo Tahiti
atggcacccaacaaatggctctaccctcgcaactgagtcctgggatattgttgattttgttattgtagtgatttaacaccccttaagattcaagatgctaagccactgcccagaagactcaagttcttgatcaaacaagatgcagaagaaaccaagattagatatcaagttcccaactcaattccaataggctctagatctaatgattaatatcacagtgttttaacctaaacaagaaacaacacacaagaacagaatcttacgtggttcaattgtgcattacatgcacaatttacattcacgggaaaagccacaggaaaagc

SEQ ID NO: 21 - DNA, 321 bp, Citrus sp.
  source 1..321: mol_type = genomic DNA; note = Pumelo_Deep_Red
atggcacccaacaaatggctctaccctcgcaactgagtcctgggatattgttattttgttattgtagtgatttaacaccccttaagattcaagatgctaagccactgcccagaagactcaagttcttgatcaaacaagatgcagaagaaaccaagattagatatcaagttcccaactcaattccaataggctctagatctaatgagtaacatttagtgttttaacctaaacaagaaacaacacacaagaacagaatcttacgtggttcaattgtgcattacatgcacaatttacattcacgggaaaagccacaggaaaagc

SEQ ID NO: 22 - DNA, 344 bp, Citrus sp.
  source 1..344: mol_type = genomic DNA; note = Pumelo_Hanna
atggcaccaacaaatggctctaccctcgcaactgagtcctgggatattgttgattttgttattgtagtgatttaacaccccttaagattcaagatgctaagccactgcccagaagactcaagttcttgatcaaacaagatgcagaagaaaccaagattagatatcaagttcccaagattagatatcaagttcccaactcaattccaataggctctagatctaatgattaatatcacagtgttttaacctaaacaagaaacaacacacaagaacagaatcttacgtggttcaattgtgcattacatgcacaatttacattcacgggaaaagccacaaggaaaagc

SEQ ID NO: 23 - DNA, 324 bp, Citrus sp.
  source 1..324: mol_type = genomic DNA; note = Pumelo_Melogold
atggcacccaaacaaatggctctaccctcgcaactgagtcctgggatattgttgattttgttattgtagtgatttaacaccccttaagattcaagatgctaagccactgcccagaagactcaagttcttgatcaaacaagatgcagaagaaaccaagattagatatcaagttcccaactcaattccaataggctctagatctaatgattaatatcacagtgttttaacctaaacaagaaacaacacacaagaacagaatcttacgtggttcaattgtgcattacatgcacaatttacattcacgggaaaagccacaggaaaagc

SEQ ID NO: 24 - DNA, 322 bp, Citrus sp.
  source 1..322: mol_type = genomic DNA; note = Pumelo_Acidless
atggcaccaacaaatggctctaccctcgcaactgagtcctgggatattgttgattttgttattgtagtgatttaacaccccttaagattcaagatgctaagccactgcccagaagactcaagttcttgatcaaacaagatgcagaagaaaccaagattagatatcaagttcccaactcaattccaataggctctagatctaatgattaatatcacagtgttttaacctaaacaagaaacaacacacaagaacagaatcttacgtggttcaattgtgcattacatgcacaatttacattcacgggaaaagccacaggaaaagc

SEQ ID NO: 25 - DNA, 322 bp, Citrus sp.
  source 1..322: mol_type = genomic DNA; note = Pumelo_Goliath
atggcaccaacaaatggctctaccctcgcaactgagtcctgggatattgttgattttgttattgtagtgatttaacaccccttaagattcaagatgctaagccactgcccagaagactcaagttcttgatcaaacaagatgcagaagaaaccaagattagatatcaagttcccaactcaattccaataggctctagatctaatgattaatatcacagtgttttaacctaaacaagaaacaacacacaagaacagaatcttacgtggttcaattgtgcattacatgcacaatttacattcacgggaaaagccacaggaaatgc

SEQ ID NO: 26 - DNA, 1092 bp, Citrus maxima
  source 1..1092: mol_type = genomic DNA
atggcaccaacaaatggctctaccctcgcaactgagtcctgggatattgctgattttgttataaacaaaggccatggagtgaagggtctctccgaaatgggactcaaaagcctcccaaaacaatttcaccaacccttggaagaaagattcagtgagaagaaaatcctggatcaagtatcaattcccctcattgatatgtcaaactgggagagtcctgaagtcgcaaaatccatttgcgatgccgctgagaattggggatttttccagattgtgaatcatggtgtgcccctggaagtactcgagagagtaaaggaagcgacccacaggtttttcgcgttaccggcagaggaaaagaggaagtactcaaaagagaattcaccaaccaataacgtgaggttcggctcaagctttgttcctcatgtggaaaaggctttggagtggaaagactttctcagcctattttatgtttctgaggaagaaacttctgcattttggcctcctgtttgcaaggatgaaatgttggagtacatgaagagttctgaggttctcattagacgtttaatgcatgtgctagtaaaggggctaaatgtgaaacgaattgatgaaataagagaacctatgttgttgggttcaagaagagttaacctcaactactatcctatgtgtcccaatcctgaactcacagtgggagtaggccgtcattcagatatttcaacattcaccatcctcctccaggatgatatcgggggacttcatgtgcggaaagacagtggtaatgactggattcatgtcgctccaattagcggatcactcattatcaacattggtgatgcattacaaataatgagcaatggtcgatacaaaagtattgagcattgtgtgattgccaatggaagccagaacaggatttcggtcccccttttcgtgaacccaaaacctgaagccatactctgtccttttcccgaagtgctcgcaaatggagagaagccactgtataagccagttttgtgtgcagactattccagacatttctatacgaaagcacatgatggaaagaaaactatcgactttgcaaagataggtgacttttga

SEQ ID NO: 27 - protein, 363 aa, synthetic construct
  REGION 1..363: note = Single strand DNA oligonucleotide
MAPTNGSTLATESWDIADFVINKGHGVKGLSEMGLKSLPKQFHQPLEERFSEKKILDQVSIPLIDMSNWESPEVAKSICDAAENWGFFQIVNHGVPLEVLERVKEATHRFFALPAEEKRKYSKENSPTNNVRFGSSFVPHVEKALEWKDFLSLFYVSEEETSAFWPPVCKDEMLEYMKSSEVLIRRLMHVLVKGLNVKRIDEIREPMLLGSRRVNLNYYPMCPNPELTVGVGRHSDISTFTILLQDDIGGLHVRKDSGNDWIHVAPISGSLIINIGDALQIMSNGRYKSIEHCVIANGSQNRISVPLFVNPKPEAILCPFPEVLANGEKPLYKPVLCADYSRHFYTKAHDGKKTIDFAKIGDF

SEQ ID NO: 28 - protein, 9 aa, synthetic construct
  REGION 1..9: note = meganucleases conserved motif
LAGLIDADG

SEQ ID NO: 29 - DNA, 125 bp, Citrus grandis
  source 1..125: mol_type = genomic DNA
atggcaccaacaaatggctctaccctcgcaactgagtcctgggatattgctgattttgttataaacaaaggccatggagtgaagggtctctccgaaatgggactcaaaagcctcccaaaacaatt

SEQ ID NO: 30 - DNA, 125 bp, Citrus clementina; identical to SEQ ID NO: 29
  source 1..125: mol_type = genomic DNA

SEQ ID NO: 31 - DNA, 780 bp, Citrus reticulata
  source 1..780: mol_type = genomic DNA
atggcaccaacaaatggctctaccctcgcaactgagtcctgggatattgctgattttgttattgtagtgatttaacaccccttaagactcaagatgctaagtcactgcccagaagactcaagttcttgatcaaacaagatgcagaagaaaccaagattagatatcaagttcccaactcaattccaataggctctagatctaatgattaatatcacagtgttttaacctaaacaagaaacaacacacaagaacagaatcttacgtggttcaattgtgcattacatgcacaatttacatccacgggaaaagccacaggaaaagctttattgatgaatcaagaagacaaatacagagcttctaaacaagagattacaagcaacaagaataacctctctcactttctgatctctctctcaagctctctcagaaccacgtctgtttctccctcagcgaccagttttacaccactgatgctgaaacctagattgctcagaatctggacacgtgtgtaacggcttttaacagccttgattcgcttcttcacgagctggccacgtgcttcacttaagttcatccaaaacgacagctgctgagtcgtttagcctttgaacttaaaaacctcgtgctgcacgtgacaaatgactgaacaatttgatcaaaacgaaaccgttttgggactctgagttttcgattcattgcactcaagtccacagttataaacaaaggtcttggagtgaagggtctctccgaaatgggactcaaaagcctcccaaaacaatt

SEQ ID NO: 32 - DNA, 780 bp, Citrus sinensis
  source 1..780: mol_type = genomic DNA
atggcaccaacaaatggctctaccctcgcaactgagtcctgggatattgttgattttgttattgtagtgatttaacaccccttaagattcaagatgctaagccactgcccagaagactcaagttcttgatcaaacaagatgcagaagaaaccaagattagatatcaagttcccaactcaattccaataggctctagatctaatgattaatatcacagtgttttaacctaaacaagaaacaacacacaagaacagaatcttacgtggttcaattgtgcattacatgcacaatttacattcacgggaaaagccacaggaaaagctttattgatgaatcaagaagacaaatacagagcttctaaacaagagattacaagcaacaagaataacctctctcactttctgatctctctctcaagctctctcagaaccacgtctgtttctccctcagcgaccagttttataccactgatgctgaaacccagattgctcagaatctggacacgtgtgtaacggtttttaacagccttgattcgcttcttcacgagctggccacgtgcttcacttaagttcatccaaaacgacagctgctgagttgtttagcctttgaacttaaaaacctcgtgctgcacgtgacaaatgactgaacaatttgatcaaaacgaaaccgttttgggactctgagttttcgattcattgcactcaagtccacagttataaacaaaggtcatggagtgaagggtctctccgaaatgggactcaaaagcctcccaaaacaatt

SEQ ID NO: 33 - DNA, 650 bp, synthetic construct
  misc_feature 1..650: note = DNA allignment product
  source 1..650: mol_type = other DNA
tgtagtgatttaacaccccttaagattcaagatgctaagccactgcccagaagactcaagccaataggctctagatctaatgattaatatcacagtgttttaacctaaacaagaaacaacacacaagaacagaatcttacgtggttcaattgtgcattacatgcacaatttacattcacgggaaaagccacaggaaaagctttattgatgaatcaagaggacaaatacagagcttctaaacaagagattacaagcaacaagaataacctctctcactttctgatctctctctcaagctctctcagaaccacgtctgtttctccctcagcgaccagttttataccactgatgctgaaacccagattgctcagaatctggacacgtgtgtaacggcttttaacagccttgattcgcttcttcacgagctggccacgtgcttcacttaagttcatccaaaacgacagctgctgagttgtttagcctttgaacttaaaaacctcgtgctgcacgtgacaaatgactgaacaatttgatcaaaacgaaaccgttttgggactctgagttttcgattcattgcactcaagtccacttcttgatcaaacaagatgcagaagaaaccaagattagatatcaagttcccaactcaatta

SEQ ID NO: 34 - DNA, 659 bp, synthetic construct
  misc_feature 1..659: note = DNA allignment product
  source 1..659: mol_type = other DNA
tgtagtgatttaacaccccttaagactcaagatgctaagccactgcccaggagactcaagttcttgatcaaacaagttgcagaagaaaccaagattagatatctagttctcaactcaatcccaataggctctagatctaatgattaatatcagtgtttataacctaaacaagaaacaattcacaacacacaagaaacagaatattacgtggttcaattgtgcattacatgcacaatttacatccacgggaaaagccacaggaaaagctttattgatgaatcaagaagacaaatacagagcttctcaacaagagattacaagcaacaagattacactctctcactttctgatctctctctcaagctctctctgaaccacacgtatttctccctcagcaaccagttttatacccctgatgctgaaacccagattgcacagaatctggacacgtgtgtaacggcttttaacagccttgattcctttcttcacgagctggctcacgtgcttcacttaagtttctccaaaacgacagatgctgagtcgtttagcctttgaacttaaaagccacgtgctgcacgtgacaaatgactgaatcaatttgatcaaaacgaagccgttttgggactctgagttttggattcattgcactcaagtccaca

SEQ ID NO: 35 - DNA, 650 bp, synthetic construct; identical to SEQ ID NO: 33
  misc_feature 1..650: note = DNA allignment product
  source 1..650: mol_type = other DNA

SEQ ID NO: 36 - DNA, 653 bp, synthetic construct
  misc_feature 1..653: note = DNA allignment product
  source 1..653: mol_type = other DNA
tgtagtgatttaacaccccttaagattcaagatgctaagccactgcccagaagactcaagccaataggctctagatctaatgattaatatcacagtgttttaacctaaacaagaaacaacacacaagaacagaatcttacgtggttcaattgtgcattacatgcacaatttacattcacgggaaaagccacaggaaaagctttattgatgaatcaagaggacaaatacagagcttctaaacaagagattacaagcaacaagaataacctctctcactttctgatctctctctcaagctctctcagaaccacgtctgtttctccctcagcgaccagttttataccactgatgctgaaacccagattgctcagaatctggacacgtgtgtaacggcttttaacagccttgattcgcttcttcacgagctggccacgtgcttcacttaagttcatccaaaacgacagctgctgagttgtttagcctttgaacttaaaaacctcgtgctgcacgtgacaaatgactgaacaatttgatcaaaacgaaaccgttttgggactctgagttttcgattcattgcactcaagtccacttcttgatcaaacaagatgcagaagaaaccaagattagatatcaagttcccaactcaattagtt

SEQ ID NO: 37 - DNA, 655 bp, synthetic construct
  source 1..655: mol_type = other DNA; note = Copia-like LTR retrotransposon
tgtagtgatttaacaccccttaagactcaagatgctaagtcactgcccagaagactcaagttcttgatcaaacaagatgcagaagaaaccaagattagatatcaagttcccaactcaattccaataggctctagatctaatgattaatatcacagtgttttaacctaaacaagaaacaacacacaagaacagaatcttacgtggttcaattgtgcattacatgcacaatttacatccacgggaaaagccacaggaaaagctttattgatgaatcaagaagacaaatacagagcttctaaacaagagattacaagcaacaagaataacctctctcactttctgatctctctctcaagctctctcagaaccacgtctgtttctccctcagcgaccagttttacaccactgatgctgaaacctagattgctcagaatctggacacgtgtgtaacggcttttaacagccttgattcgcttcttcacgagctggccacgtgcttcacttaagttcatccaaaacgacagctgctgagtcgtttagcctttgaacttaaaaacctcgtgctgcacgtgacaaatgactgaacaatttgatcaaaacgaaaccgttttgggactctgagttttcgattcattgcactcaagtccacagttat

SEQ ID NO: 38 - DNA, 1257 bp, Citrus limon
  source 1..1257: mol_type = genomic DNA
atggcaccaactaatggctctatcctcgcaactgagtcctgggatattgctgattttgttataaacaaaggtcatggagtgaagggtctctctgaaatgggactcaaaagcctccaaaaacaatttcaccaacccttagaagaaagaatcagtgagaagaaaatcttgggtcaagtatcaattcccctcattgataagttaaaatgggagagtcctgaagtcgcaaaatccatttgcgatgccgctgagagttggggatttttccagattgtgaatcatggtgtgcccctggaagtactcgagagagtaaaggaagcgacccacaggtttttcgcgttaccggcagaggagaagaggaagtactcaaaagagaattcaccaaccaataaagtgaggttcggctcaagctttgttcctcatgtggaaaaggctttggagtggaaagactttctcagcctattttatgtttctgaggaagaaacttctgcattttggcctcctgtttgcaaggatgaaatgttggagtacatgaagagttctgaggttctcattagacgtttaatgcatgtgctagtcaaggggctaaatgtgaaacgaattgatgaaataagagaacctatgttgttgggttcaagaagagttaacctcaactactatcctatgtgtcccaatcctgaactcacagtgggagtaggccgtcattcagatatttcaacattcaccatcctcctccaggatgatatcgggggacttcatgtgcggaaagacagtggtaatgactggattcatgtcgctccaattagcggatcactcattatcaacattggtgatgcattacaaataatgagcaatggtcgatacaaaagtattgagcattgtgtgattgccaatggaagccagaacaggatttcggtcccccttttcgtgaacccaaaacctgaagccatactctgtccttttcccgaagtgctcgcaaatggagagaagccactgtataagccagttttgtgtgcagactattccagacatttctatacgaaagcacatgatggaaagaaaactatcgactttgcaaagataggttggtttcctagaataatcccgaaaccatttcttcactcgtgggtatcaaagataatgggctttactctagtttcaacagtttatgtattaaattatttcaataaattttatttcactctactgggagtaattcatccttctctctctcatcctattgattattccataaattaa

SEQ ID NO: 39 - protein, 418 aa, Citrus limon
MAPTNGSILATESWDIADFVINKGHGVKGLSEMGLKSLQKQFHQPLEERISEKKILGQVSIPLIDKLKWESPEVAKSICDAAESWGFFQIVNHGVPLEVLERVKEATHRFFALPAEEKRKYSKENSPTNKVRFGSSFVPHVEKALEWKDFLSLFYVSEEETSAFWPPVCKDEMLEYMKSSEVLIRRLMHVLVKGLNVKRIDEIREPMLLGSRRVNLNYYPMCPNPELTVGVGRHSDISTFTILLQDDIGGLHVRKDSGNDWIHVAPISGSLIINIGDALQIMSNGRYKSIEHCVIANGSQNRISVPLFVNPKPEAILCPFPEVLANGEKPLYKPVLCADYSRHFYTKAHDGKKTIDFAKIGWFPRIIPKPFLHSWVSKIMGFTLVSTVYVLNYFNKFYFTLLGVIHPSLSHPIDYSIN

(19) United States
(12) Patent Application Publication
(10) Pub. No.: US 2026/0218219 A1
(43) Pub. Date: Jul. 30, 2026

(54) CITRUS PLANTS WITH MODIFIED PRODUCTION OF COUMARINS AND FURANOCOUMARINS METHODS OF PRODUCING SAME AND PRODUCTS OBTAINED THEREBY

(71) Applicant: The State of Israel, Ministry of Agriculture & Rural Development, Agricultural Research Organization, Rishon-LeZion (IL)

(72) Inventors: Yoram EYAL, Mesilat Zion (IL); Livnat GOLDENBERG, Rehovot (IL); Nir CARMI, Rehovot (IL); Amir SHERMAN, Beit Zayit (IL); Ron OPHIR, Modi'in Makabim-Re'ut (IL)

(73) Assignee: The State of Israel, Ministry of Agriculture & Rural Development, Agricultural Research Organization, Rishon-LeZion (IL)

(21) Appl. No.: 19/141,389

(22) PCT Filed: Dec. 21, 2023
(86) PCT No.: PCT/IL2023/051294

§ 371 Date: Jun. 20, 2025

Related U.S. Application Data
(60) Provisional application No. 63/434,558, filed on Dec. 22, 2022.

(51) Int. Cl. C12N 15/82

(52) U.S. Cl. CPC C12N 15/8243

(57) ABSTRACT

A citrus plant is provided. The plant comprises a genome having a loss of function mutation in a Cg2g000710 gene or ortholog thereof, wherein the citrus plant is a natural producer of coumarin or furanocoumarin (FC). Also provided are methods of producing such plants.

### RELATED APPLICATION/S

This application claims priority from U.S. Provisional Patent Application No. 63/434,558 filed on Dec. 22, 2022 which is hereby incorporated by reference in its entirety.

### SEQUENCE LISTING STATEMENT

The XML file, entitled 98820.xml, created on Dec. 18, 2023, comprising 54,895 bytes, submitted concurrently with the filing of this application is incorporated herein by reference.

### FIELD AND BACKGROUND OF THE INVENTION

The present invention, in some embodiments thereof, relates to citrus plants with modified production of coumarins and furanocoumarins methods of producing same and products obtained thereby.

Furanocoumarins (FCs) are a class of bioactive polyphenolic plant defense compounds derived from the phenylpropanoid pathway via the coumarin umbelliferon (UM), and are associated with hazards to human health. Ingested FCs common in grapefruit and pumelo have been implicated as a major factor leading to prescription-drug overdose, as documented in the well-studied “grapefruit-drug interactions”¹ involving inhibition of the intestinal/liver drug-detoxifying enzyme CYP3A4 by FC compounds abundant in grapefruit². In addition, the transport of other prescription drugs into the bloodstream and the target cells may be inhibited by FCs leading to an ineffective concentration^{3,4}. Accordingly, consumption of grapefruit is forbidden for the users of a wide range of prescription drugs, including the commonly used statins^{2,5-7}. An additional health-hazard involving FCs is phytophotodermatitis, a potentially severe form of dermatitis resulting from accumulation of DNA-binding FCs in skin cells followed by exposure to UVA from sunlight⁸⁻¹⁰. Plant species implicated in potential involvement in phytophotodermatits belong to the families: Apiaceae (celery, parsley, parsnip), Rutaceae (bergamot orange, lime, common rue), and Moraceae (common fig)^{8,11-15}.

While FCs may contribute nutritionally as antioxidants and anti-inflammatory agents¹⁶, and they clearly have a significant role in plant defense bearing allelopathic and antifungal attributes¹⁷, their negative effects from a human standpoint appear to outweigh their potential positive roles in edible plant tissues. The repercussions of exposure or ingestion of FCs to human health underscore the importance of elucidating the FC biosynthetic pathway and respective genes towards a long-term goal of developing plant varieties harboring FC-free edible plant tissues. Accordingly, the FC biosynthetic pathway has attracted extensive interest in recent years and its steps have been mostly worked out based on the identification of intermediates in the pathway^{11,17,18}. The enzyme p-coumaroyl CoA 2-hydroxylase (C2′H), which is a member of the 2-oxoglutarate-dependent dioxygenase family (2OGDs), catalyzes synthesis of umbelliferon, a committed step in coumarin biosynthesis that also serves as the entry point for furanocoumarin biosynthesis (FIG. 1). A gene encoding C2′H was characterized from common rue (Ruta graveolens), sweet potato (Ipomoea batatas), parsnip (Pastinaca sativa) and Peucedanum praeruptorum^{15,19-21}. Downstream of umbelliferon, many of the enzymatic steps providing the wide repertoire of FCs are predicted to be catalyzed by prenyltransferases or by members of the cytochrome P450 family, and several were characterized at the encoding gene level in different plants; parsley (Petroselinum crispum), parsnip (P. sativa), fig (Ficus carica) and Citrus^{11-13,18}. Nevertheless, many of the pathway enzymes and corresponding genes have not been characterized as yet, and since at least some of the FC biosynthetic apparatus appears to have evolved in parallel in different plant families (i.e., convergent evolution)^{11,18,22}, identifying orthologous genes between distant plant species merely based on sequence similarity is not effective.

The genus Citrus contains species/varieties which are among the highest FC producers amid consumed plant species, including pumelo (Citrus grandis), grapefruit (C. paradisi), lime (C. aurantiifolia) and sour orange (C. aurantium). Grapefruit is considered as the major source of dietary FCs in the western world and contains high levels of the linear FC branch including bergamottin and 6′,7′-dihydroxybergamottin (6,7-DHB), which are well established players in the grapefruit-drug interactions^{5,23} (FIG. 1). Citron (C. medica) and lemon (C. limon) contain moderate-low FC levels, while mandarin (C. reticulata), clementine (C. clementina) and sweet orange (C. sinensis) have very low to undetectable FC levels²⁴ and are considered safe for consumption by prescription-drug users.

### SUMMARY OF THE INVENTION

According to an aspect of some embodiments of the present invention there is provided a citrus plant comprising a genome having a loss of function mutation in a Cg2g000710 gene or ortholog thereof, wherein the citrus plant is a natural producer of coumarin or furanocoumarin (FC).

According to some embodiments of the invention, the plant is selected from the group consisting of pumelo (Citrus grandis), grapefruit (C. paradisi), lime (C. aurantiifolia) and sour orange (C. aurantium).

According to some embodiments of the invention, the plant is selected from the group consisting of citron (C. medica) and lemon (C. limon).

According to some embodiments of the invention, the loss of function mutation is in both alleles of the genome.

According to some embodiments of the invention, the loss of function mutation is in a homozygous form.

According to some embodiments of the invention, the coumarin is selected from the group consisting of scopoletin and umbelliferon.

According to some embodiments of the invention, the FC is selected from the group consisting of bergamottin, 6′,7′-dihydroxybergamottin (6,7-DHB), marmesin, psoralen and bergaptol.

According to some embodiments of the invention, the loss of function mutation is selected from the group consisting of an insertion, a deletion, an insertion/deletion (indel) and a substitution.

According to some embodiments of the invention, the loss of function mutation is an insertion.

According to some embodiments of the invention, the insertion is of a transposon sequence.

According to some embodiments of the invention, the transposon sequence is as set forth in SEQ ID NO: 37 (Copia-like LTR retrotransposon).

According to some embodiments of the invention, the loss of function mutation affects a C2′H and/or F6′H activity of the gene.

According to some embodiments of the invention, herein the plant part is fruit, cutting or seed.

According to an aspect of some embodiments of the present invention there is provided a part of the plant as described herein.

According to an aspect of some embodiments of the present invention there is provided the plant part is a fruit.

According to an aspect of some embodiments of the present invention there is provided a processed product of the fruit as described herein.

According to an aspect of some embodiments of the present invention there is provided a method of producing an edible product, comprising processing the fruit as described herein.

According to an aspect of some embodiments of the present invention there is provided a method of producing the plant as described herein, the method comprising:
(a) providing a citrus plant that is a natural producer of FC;
(b) genetically modifying a Cg2g000710 gene or ortholog thereof so as to result in a loss of function mutation in the gene of ortholog thereof.

According to some embodiments of the invention, the genetically modifying is by genome editing.

According to some embodiments of the invention, the genetically modifying is by breeding with a citrus plant which is a non-natural producer of coumarin or FC.

According to some embodiments of the invention, the genetically modifying is in both alleles of the gene or ortholog thereof.

According to some embodiments of the invention, the method further comprises validating loss of function of the Cg2g000710 gene or ortholog thereof.

According to some embodiments of the invention, the validating is effected using molecular markers.

According to some embodiments of the invention, the validating is effected biochemically by testing a function of the Cg2g000710 gene or ortholog thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of the biosynthetic pathway from phenylalanine to linear furanocoumarins involved in “Grapefruit-drug interactions”. PAL—phenylalanine ammonia lyase; C4H—cinnamic acid 4-hydroxylase; 4CL—4-coumaroyl CoA ligase; C3H—coumaroyl CoA 3-hydroxylase; OMT—O-methyl transferase; F6′H—feruloyl CoA 6′-hydroxylase; C2′H—p-coumaroyl CoA 2′-hydroxylase; PT—prenyl transferase; P450—cytochrome P450. Black arrows indicate single direct reaction, dashed arrows represent several reactions.

FIG. 2 is a graphic presentation of the differential production of furanocoumarins in ‘Ora’ X ‘Hudson’ population. Umbelliferon and furanocoumarin content in the young leaves of parental lines ‘Ora’ mandarin and ‘Hudson’ grapefruit, and of their ‘None-producing’ and ‘FC/UM’ producing F1 progeny. Shown are means±SE of three biological replica quantified by quantitative LC/MS and presented as nanogram product per-gram fresh weight.

FIG. 3 is an illustration showing that Cg2g000710 is intact in pumelo and its orthologue is disrupted in mandarin. Schematic representation of the gene Cg2g000710 in pumelo (C. grandis) and its orthologue MSYJ070210 in mandarin (C. reticulata). Straight lines indicate non-coding sequences, filled boxes indicate exons and empty dashed-line boxes indicate interrupted coding sequences. Levels of umbelliferon and/or furanocoumarins in leaves and/or fruit (FCs/UM) are indicated. N.D.=not detected.

FIG. 4 shows that C2′H/F6′H is involved in both umbelliferon and scopoletin production. In-vivo activity assay of Cg_C2H/F6H (Cg2g000710 encoded enzyme) in N. benthamiana leaves. Agrobacterium clones harboring constructs for the expression of Cg_C2H/F6H coding sequence, R. graveolens positive control plasmid (JF799117.1; Rg_C2H/F6H) or ER-mCherry negative control plasmid (mCherry) were infiltrated into N. benthamiana leaves. The substrates coumaric acid (CA) or ferulic acid (FA) were infiltrated into the leaves after 48 hours and leaves were harvested and extracted after an additional 72 hours. Substrate (coumaric acid and ferulic acid) 20 and product (umbelliferon and scopoletin) levels were measured by quantitative LC/MS. Shown are means±SE of three biological replica and presented as nanogram product per-gram fresh weight.

FIGS. 5A-C show that Cg_C2H/F6H-orthologue intact allele is necessary for FCs/UM production in citrus. Correlation between Cg_C2H/F6H-orthologue alleles and the levels of umbelliferon and/or furanocoumarins in leaves and/or fruit (FCs/UM) among different citrus varieties. Two sets of primers were designed as schematically shown in A to amplify either the Cg_C2H/F6H-orthologue intact allele (‘Intact allele’) or the 655 bases insertion inactivated allele (‘Insertion allele’) by PCR. A, Distribution of the Cg_C2H/F6H-orthologue intact and insertion inactivated alleles in parental lines ‘Ora’ mandarin and ‘Hudson’ grapefruit, and their ‘None-producing’ and ‘FC/UM producing’ F1 progeny. Levels of FC/UM are indicated below (+=significant FC/UM levels, ND=no FC/UM detected). B, Distribution of intact and/or insertion allele in parental lines ‘Pazit’ mandarin and ‘Chandler’ pumelo, and their F1 progeny. Levels of FC/UM are indicated below. C, Distribution of intact and/or insertion allele in citrus ancestors and hybrids, in correlation to the respective levels of FC/UM in fruit and young leaves (quantitatively shown in Dugrand-Judek, et al., 2015²⁴ and Durand-Hulak, et al., 2015²⁶).

FIGS. 6A-B show a multi-gene locus containing putative 2-oxoglutarate-dependent dioxygenases (2OGDs) of pumelo (C. grandis), and their phylogenetic relationships. A, Schematic location of the multi-gene locus of putative 2OGDs on C. grandis chromosome #2. B, Phylogenetic tree (non-rooted) of pumelo putative 2OGDs and previously characterized 2OGDs of the C2′H/F2′H subgroup: Rg_C2H_F6H (R. graveolens)—ADV77970.1, Ib_C2H_F6H (I. batatas)—AB636149, Ps_C2H (P. sativa)—APP 94171.1, Pp_C2H (P. praeruptorum)—ASR80916.1, At_F6H (A. thaliana)—AAS49108.1. Bootstrap values are indicated.

FIGS. 7A-E shown that the Citrus 2OGD multi-gene locus regulates organ-specific furanocoumarin biosynthesis. Tissue-specific gene expression levels of the three highly similar 2OGDs and the respective furanocoumarins/coumarins levels. A, 2OGDs gene expression in young leaves of parental lines ‘Ora’ mandarin and ‘Hudson’ grapefruit, presented as FPKM (fragments per kilo base of transcript per million mapped fragments). B, gene expression of the 2OGDsin the fruitlets of parental lines ‘Ora’ mandarin and ‘Hudson’ grapefruit, presented as fold change (quantitative RT-PCR). C, gene expression of the 2OGDs in roots of parental line ‘Hudson’ grapefruit, and ‘Willowleaf’, ‘Cleopatra’ and ‘Murcott’ mandarins, presented as fold change (quantitative RT-PCR). D, Total umbelliferon and furanocoumarin levels in young leaves, fruitlets and roots tissues, presented as nanogram product per-gram fresh weight. E, Comparative expression of the three putative 2OGDs in leaf/fruit/root tissues of ‘Hudson’ grapefruit (quantitative RT-PCR). In all charts means±SE of three biological replica are shown. Asterisks indicate significant difference between/among varieties (P<0.05, Student's t-test and/or Tukey-Kramer test).

FIGS. 8A-C show a tissue specific differential furanocoumarins composition in citrus. FC/UM and scopoletin levels in young leaves of parental lines ‘Ora’ and ‘Hudson’ varieties (a), in fruitlets of ‘Ora’ and ‘Hudson’ varieties (b), and in roots of parental line ‘Hudson’ grapefruit, and ‘Willowleaf’, ‘Cleopatra’ and ‘Murcott’ mandarins (c). Values are presented as nanogram product per-gram fresh weight. Shown are means±SE of three biological replica. Asterisks indicate significant difference between ‘Ora’ and ‘Hudson’ varieties (P<0.05, Student's t-test).

FIG. 9 shows that the ORF of Cg2g000710 orthologues (SEQ ID Nos: 29-32) is disrupted by a 655 base insertion sequence. ClustalW nucleotide alignment of the putative Cg_2OGD from pomelo (C. grandis; Cg2g000710), and the orthologous genes from mandarin (C. reticulata; MSYJ070210), clementine (C. clementina; Ciclev10017914m) and sweet orange (C. sinensis; Cs9g02910) (www(dot)citrusgenomedb(dot)org/). First coding ATG is highlighted in green, protein coding sequence is highlighted in yellow, 655 bases insertion sequence is marked with red letters, stop codons are highlighted in red, and intron is marked with blue letters.

FIG. 10 shows the ruling out spontaneous conversion of scopoletin to umbelliferon in the plant tissue. Umbelliferon, scopoletin and their substrates levels after infiltration of mCherry control plasmid, followed by additional infiltration after 48 h of umbelliferon and scopoletin to N. benthamiana leaves. Leaves were extracted after a total of 5 days. Shown are means±SE of three replica quantified by quantitative LC/MS.

FIG. 11 shows pumelo varieties genomic DNA contain the ‘Insertion allele’. Sequencing results of PCR products of 6 pumelo varieties, using primers flanking CDS/insertion junction of Cg2g000710 gene (SEQ ID NO: 19-25). Primers sequences are highlighted in yellow, base pairs insertions in the primer region comparing to C. reticulata sequence are highlighted in red, base pairs substitutions in the primer region comparing to C. reticulata sequence are marked with red letters.

FIG. 12 shows that the Pumelo varieties genomic DNA contain the ‘Insertion allele’. Sequencing results of PCR products of 6 pumelo varieties, using primers flanking CDS/insertion junction of Cg2g000710 gene (SEQ ID Nos: 33-36). Primers sequences are highlighted in yellow, base pairs insertions in the primer region comparing to C. reticulata sequence are highlighted in red, base pairs substitutions in the primer region comparing to C. reticulata sequence are marked with red letters.

### DESCRIPTION OF SPECIFIC EMBODIMENTS OF THE INVENTION

The present invention, in some embodiments thereof, relates to citrus plants with modified production of coumarins and furanocoumarins methods of producing same and products obtained thereby.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Furanocoumarins (FCs) are a class of bioactive polyphenolic plant defense compounds derived from the phenylpropanoid pathway via the coumarin umbelliferon (UM), and are associated with hazards to human health. Ingested FCs common in grapefruit and pumelo have been implicated as a major factor leading to prescription-drug overdose, as documented in the well-studied “grapefruit-drug interactions”. In addition, the transport of other prescription drugs into the bloodstream and the target cells may be inhibited by FCs leading to an ineffective concentration^{3,4}.

Whilst conceiving and reducing embodiments of the invention to practice, the present inventors identified a multiple gene locus involved in FC biosynthesis in citrus, one of the genes being outstanding in its role in production of coumarins in citrus leaves and fruit. Some embodiments of the invention relate, therefore, to targeting the gene Cg2g000710 or orthologs thereof in natural producers of coumarins and FCs and to use of same in molecular marker applications.

As is shown hereinbelow and in the Examples section which follows, the present inventors sought to identify the key genes in the coumarins and FCs synthetic pathway and to understand the genetic basis for the difference between citrus FC producers (grapefruit, pumelo) and non-producers (mandarin, orange). They used a genetic approach based on mandarin-grapefruit and mandarin-pumelo F1 populations. Metabolite analysis of the parental lines and F1 populations suggested that lack of FCs in mandarin fruit/leaves is due to a block in umbelliferon biosynthesis. Transcriptome and genome analyses established a strong correlation of the metabolite profile with integrity of a leaf/fruit-specific 2OGD-family gene/enzyme, Cg2g000710, that catalyzes biosynthesis of umbelliferon; while grapefruit and pumelo possess at least one intact allele of the gene, mandarin and orange are homozygous for a solo-LTR insertion-sequence disrupting the 2OGD-family gene coding region. Transient gene expression in Nicotiana benthamiana together with substrates (p-coumaroyl CoA or Feruloyl CoA) proved that Cg2g000710 encodes a C2′H/F6′H enzyme catalyzing biosynthesis of the coumarins umbelliferon and scopoletin. Expression studies of additional 2OGD-family genes within the same genome locus suggest that a second gene directs FC biosynthesis in citrus roots, albeit resulting in a very different FC profile than fruit/leaves.

Thus, according to an aspect of the invention there is provided a citrus plant comprising a genome having a loss of function mutation in a Cg2g000710 gene or ortholog thereof, wherein the citrus plant is a natural producer of coumarin or furanocoumarin (FC).

As used herein l “a natural producer of coumarin or furanocoumarin (FC)” refers to a citrus plant species which produces coumarin or furanocoumarin (FC) in the fruit, at a level above ~40 mmol·Kg⁻¹ FW in the pulp and above ~200 mmol·Kg⁻¹ FW in the peel as can be determined by LC-MS analyses (Dugrand-Judek et al., 2015).

According to a specific embodiment, the coumarin or FCs are present in the pulp and peel.

TABLE 1
High producers

| | Gene | Abundant | | |
|---|---|---|---|---|
| | Accession No | coumarins | | |
| | (ortholog of | and their | Abundant FCs | Examples |
| Species | Cg2g000710) | levels | and their levels | of varieties |
| Citrus | Cg2g000710 | | 6′,7′- | ‘Chandler’ |
| maxima | | | Dihydroxybergamottin | ‘Tahiti’ |
| (Pumelo) | | | Bergamottin | ‘Deep-red’ |
| | | | | ‘Goliath’ |
| Citrus | The sequence | | 6′,7′- | ‘Hudson’ |
| paradisi | of the coding | | Dihydroxybergamottin | ‘Marsh’ |
| (grapefruit) | region in | | Bergamottin | ‘Ruby red’ |
| | grapefruit is | | | ‘Star ruby’ |
| | identical to | | | |
| | Cg2g000710 | | | |
| | (pumelo). | | | |

TABLE 2
Moderate producers

| | | Abundant | | |
|---|---|---|---|---|
| | | coumarins | | Examples |
| | Gene Accession No | and their | Abundant FCs | of |
| Species | (ortholog of Cg2g000710) | levels | and their levels | varieties |
| Citrus | CL2G003535011.t1_pri_Citrus_limon_v1 | | oxypeucedanin | |
| limon | ID = CL2G003535011.t1_pri_Citrus_limon_ | | phellopterin, | |
| (lemon) | v1; Name = CL2G003535011.t1_pri (SEQ | | byakangelicol | |
| | ID Nos: 38-39) | | byakangelicin | |

Such species can be further divided to “high producers”, which produce above ~120 mmol·Kg⁻¹ FW in the pulp and “moderate producers”, which produce above 40 mmol·Kg⁻¹ FW in the pulp and up to 120.

In a non-natural producer of coumarin or furanocoumarin (FC) the level of coumarin or furanocoumarin (FC) in the pulp is usually undetectable, or below 40 mmol·Kg⁻¹ FW (e.g., below 20 or 10 mmol·Kg⁻¹ FW). Note that the fruit produces FCs at a very early developmental stage—i.e., fruitlets).

According to a specific embodiment, the non-producers are homozygous for a 655 base insertion (SEQ ID NO: 37 or of a sequence at least 95%, 96%, 97%, 98%, 99%, 99.5% or more identical thereto) in the Cg2g000710 gene or ortholog thereof, which appears to be a solo-LTR 28,30 derived from a Copia-like retrotransposon, which renders these citrus species non-producers.

According to a specific embodiment, the plant is selected from the group consisting of pumelo (Citrus grandis), grapefruit (C. paradisi), lime (C. aurantiifolia) and sour orange (C. aurantium).

According to a specific embodiment, the plant is selected from the group consisting of citron (C. medica) and lemon (C. limon).

As used herein “a Cg2g000710 gene” is the DNA, RNA or protein product of the Cg2g000710 gene (SEQ ID NO: 26-27).

As used herein “ortholog” refers to the homolog of the gene found in a different citrus species but related by linear descent.

Tables 1 and 2 above illustrate such homologs. See also, Mariana Limones-Mendez, Audray Dugrand-Judek, Cloé Villard, Victoire Coqueret, Yann Froelicher, Frédéric Bourgaud, Alexandre Olry, Alain Hehn, Convergent evolution leading to the appearance of furanocoumarins in citrus plants, Plant Science, Volume 292,2020,110392, www(dot)doi(dot)org/10(dot)1016/j(dot)plantsci(dot)2019(dot)110392; Munakata, R., Kitajima, S., Nuttens, A., Tatsumi, K., Takemura, T., Ichino, T., Galati, G., Vautrin, S., Bergès, H., Grosjean, J., Bourgaud, F., Sugiyama, A., Hehn, A. and Yazaki, K. (2020), Convergent evolution of the UbiA prenyltransferase family underlies the independent acquisition of furanocoumarins in plants. New Phytol, 225:2166-2182. www(dot)doi(dot)org/10(dot)1111/nph(dot)16277.

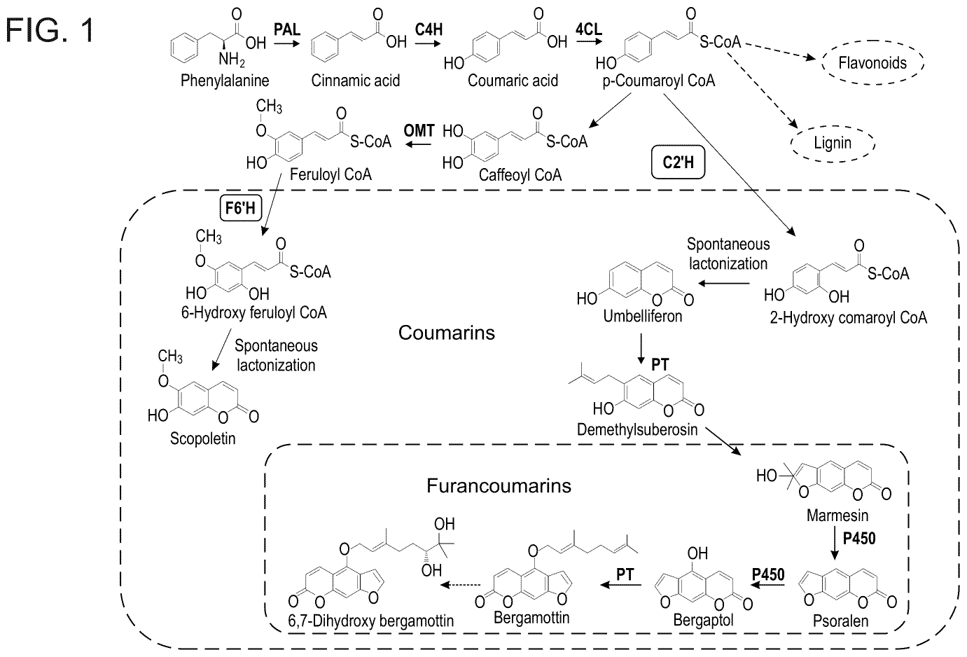

According to a specific embodiment, the gene or ortholog (herein after “the gene”) comprises the p-coumaroyl CoA 2′-hydroxylase (C2′H; EC 1.14.11.62) or feruloyl-CoA 6′-hydroxylase (F6′H; EC 1.14.11.61) that catalyze umbelliferon/scopoletin biosynthesis (see FIG. 1).

As used herein, the phrase “loss-of-function mutation” refers to at least one mutation in the DNA sequence of a gene (in this case a Cg2g000710 gene or ortholog thereof), which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity or which sentence the protein to degradation; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift. The mutation can be in a coding region or a non-coding region.

According to specific embodiments, the loss of function mutation is selected from the group consisting of an insertion, a deletion, an insertion/deletion (indel) and a substitution.

According to specific embodiments, the loss of function mutation is an insertion.

According to specific embodiments, the insertion is of a transposon sequence.

According to specific embodiments, the transposon sequence is as set forth in SEQ ID NO: 37 (Copia-like LTR retrotransposon).

According to specific embodiments, the loss of function mutation is not by an insertion of the Copia-like LTR retrotransposon in the gene.

According to specific embodiments, the loss-of-function mutation of the gene may comprise at least one allele of the gene.

It will be appreciated that since the gene is a single gene dominating the trait in order to have an adequate effect both alleles should comprise a loss of function mutation (not necessarily the same in a homozygous form), but a single allele mutation can be also useful for breeding and research hence both are contemplated herein.

According to specific embodiments, the loss of function mutation is in both alleles of the genome.

According to specific embodiments, the loss of function mutation is in a homozygous form.

The term “allele” as used herein, refers to any of one or more alternative forms of a gene locus, which alleles relate to a trait or characteristic. In a diploid citrus cell or organism, the two alleles of a given gene occupy corresponding loci on a pair of homologous chromosomes.

According to other specific embodiments a loss-of-function mutation of a gene comprises both alleles of the gene. In such instances the e.g. Cg2g000710 may be in a homozygous form or in a heterozygous form. According to this embodiment, homozygosity is a condition where both alleles of the gene e.g. Cg2g000710 are characterized by the same nucleotide sequence. Heterozygosity refers to different conditions of the gene at the e.g. Cg2g000710 gene.

According to a specific embodiment the loss of function mutation is in a homozygous or heterozygous form yet both encode for dis-functioning products.

According to a specific embodiment, the loss of function mutation refers to at least one Indel.

According to a specific embodiment, the loss of function mutation causes a premature stop codon.

According to a specific embodiment, the mutation retains the protein product but abolishes its activity e.g., C2′H and/or F6′C activity.

According to a specific embodiment, the mutation affects the level or protein expression, e.g., to such that is undetectable at the mRNA and/or protein level as can be determined by RT-PCR or Western blot.

According to a specific embodiment, the genetic modification reduces the level or expression and/or activity of the gene by at least 80% (e.g., 81%, 82%, 83%, 84%, 85%, 86%, 87%, 88%, 89%, 90%, 91%, 92%, 93%, 94%, 95%, 96%, 97%, 98%, 99% or 100%).

According to a specific embodiment, the genetic modification reduces the level of fruit coumarin or FCs by at least 80% (e.g., 81%, 82%, 83%, 84%, 85%, 86%, 87%, 88%, 89%, 90%, 91%, 92%, 93%, 94%, 95%, 96%, 97%, 98%, 99% or 100%).

The comparison can be made to an identical tissue of a citrus plant of the same genetic background and developmental stage,

The “same genetic background” refers to at least 97%, 98%, 99% or 99.9% of the genome is shared between the plant and the non-mutated plant.

According to a specific embodiment, the plant may be a transgenic plant but the transgene may not be associated with (i.e., not the cause for) coumarin and FC synthesis, as described herein. For example, the transgene may function to improve biotic stress resistance (e.g., citrus greening or HLB), pesticide resistance or abiotic stress resistance.

According to a specific embodiment, the plant is a hybrid plant or the seed is a hybrid seed, where e.g., each of the parental lines is homozygous for a loss-of-function mutation in the gene as described herein.

Methods of producing the plant as described herein may rely on the use of mutagens e.g., EMS or breeding or genetic engineering, e.g., genome editing, which is naturally a more directed method and therefore negates the need for breeding steps, a parameter that is highly valuable in citrus trees genus.

Thus, according to an aspect of the invention there is provided a method of producing the plant with a reduced coumarin and FC content, the method comprising:
(a) providing a citrus plant that is a natural producer of FC;
(b) genetically modifying a Cg2g000710 gene or ortholog thereof so as to result in a loss of function mutation in the gene of ortholog thereof.

According to a specific embodiment, the genetically modifying is by breeding with a citrus plant which is a non-natural producer of coumarin or FC.

For example, by way of cross-breeding of mandarin or sweet orange (both homozygous non-producers) with grapefruit (heterozygous FCs producer).

According to another specific embodiment, the genetically modifying is by genome editing.

According to another specific embodiment, the genetically modifying is in both alleles of the gene or ortholog thereof.

Below is a description of platform technologies for effecting knock-out (also referred to as “genome editing”) and transcriptional silencing in plants.

Methods of introducing nucleic acid alterations to a gene of interest are well known in the art (see for example Editing the CsDMR6 gene in citrus results in resistance to the bacterial disease citrus canker. Saroj Parajuli, Heqiang Huo, Fred G Gmitter, Jr, Yongping Duan, Feng Luo, Zhanao Deng. Horticulture Research, Volume 9, 2022, uhac082, WWW (dot)doi(dot)org/10(dot)1093/hr/uhac082; Generation of homozygous canker-resistant citrus in the T0 generation using CRISPR-SpCas9p. Hongge Jia and Nian Wang. Plant Biotechnology Volume 18, Issue 10, October 2020).

Following is a description of various exemplary methods used to introduce nucleic acid alterations to a gene of interest and agents for implementing same that can be used according to specific embodiments of the present invention.

Any of the below methods can be directed to any part of the gene (e.g., Cg2g000710) as long as a loss-of-function is achieved.

As used herein “target sequence” refers to the Cg2g000710 gene or ortholog DNA or RNA transcript.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8, 129,134; 8,133,697; 8,143,015; 8,143,016; 8, 148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fok1. Additionally Fok1 has the advantage of requiring dimerization to have nuclease activity and this means the specificity increases dramatically as each nuclease partner recognizes a unique DNA sequence. To enhance this effect, Fok1 nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www (dot) talendesign (dot) org). TALEN can also be designed and obtained commercially from e.g., Sangamo Biosciences™ (Richmond, CA).

Another agent capable of downregulating Cg2g000710 gene or ortholog is a RNA-guided endonuclease technology e.g. CRISPR system (that is exemplified in great details in the Examples section which follows).

As used herein, the term “CRISPR system” also known as Clustered Regularly Interspaced Short Palindromic Repeats refers collectively to transcripts and other elements involved in the expression of or directing the activity of CRISPR-associated genes, including sequences encoding a Cas9 gene (e.g. CRISPR-associated endonuclease 9), a tracr (trans-activating CRISPR) sequence (e.g. tracrRNA or an active partial tracrRNA), a tracr-mate sequence (encompassing a “direct repeat” and a tracrRNA-processed partial direct repeat) or a guide sequence (also referred to as a “spacer”) including but not limited to a crRNA sequence (i.e. an endogenous bacterial RNA that confers target specificity yet requires tracrRNA to bind to Cas) or a sgRNA sequence (i.e. single guide RNA).

In some embodiments, one or more elements of a CRISPR system is derived from a type I, type II, or type III CRISPR system. In some embodiments, one or more elements of a CRISPR system (e.g. Cas) is derived from a particular organism comprising an endogenous CRISPR system, such as Streptococcus pyogenes, Neisseria meningitides, Streptococcus thermophilus or Treponema denticola.

In general, a CRISPR system is characterized by elements that promote the formation of a CRISPR complex at the site of a target sequence (also referred to as a protospacer in the context of an endogenous CRISPR system).

In the context of formation of a CRISPR complex, “target sequence” (in this case a Cg2g000710 gene or ortholog thereof) refers to a sequence to which a guide sequence (i.e. guide RNA e.g. sgRNA or crRNA) is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. Thus, according to some embodiments, global homology to the target sequence may be of 50%, 60%, 70%, 75%, 80%, 85%, 90%, 95% or 99%. A target sequence may comprise any polynucleotide, such as DNA or RNA polynucleotides. In some embodiments, a target sequence is located in the nucleus or cytoplasm of a cell.

Thus, the CRISPR system comprises two distinct components, a guide RNA (gRNA) that hybridizes with the target sequence, and a nuclease (e.g. Type-II Cas9 protein), wherein the gRNA targets the target sequence and the nuclease (e.g. Cas9 protein) cleaves the target sequence. The guide RNA may comprise a combination of an endogenous bacterial crRNA and tracrRNA, i.e. the gRNA combines the targeting specificity of the crRNA with the scaffolding properties of the tracrRNA (required for Cas9 binding). Alternatively, the guide RNA may be a single guide RNA capable of directly binding Cas.

Exemplary gRNA sequences for the Cg2g000710 or orthologs are provided in Table 3 below.

TABLE 3

| Target | gRNA |
|---|---|
| Cg2g000710 | gRNA 1 position 68 |
| | GCTGATTTTGTTATAAACAAAGG |
| | (SEQ ID NO: 1) |
| | gRNA 2 position 310 |
| | GGAAGTACTCGAGAGAGTAAAGG |
| | (SEQ ID NO: 2) |
| | gRNA 3 position 824 |
| | GGATCACTCATTATCAACATTGG |
| | (SEQ ID NO: 3) |
| | gRNA 4 position 851 |
| | GCATTACAAATAATGAGCAATGG |
| | (SEQ ID NO: 4) |

According to some embodiments of the invention, the editing agent (e.g., gRNAs) are specific to the target (Cg2g000710) and don't have an off-target effect, e.g., Cg2g000700 and Cg2g000720.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence.

In some embodiments, the tracr sequence has sufficient complementarity to a tracr mate sequence to hybridize and participate in formation of a CRISPR complex. As with the target sequence, a complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned.

Introducing CRISPR/Cas into a cell may be effected using one or more vectors driving expression of one or more elements of a CRISPR system such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. A single promoter may drive expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron).

“Hit and run” or “in-out”-involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, transformed into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy-involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After transformation and positive selection, homologously targeted clones are identified. Next, a second targeting vector that contains a region of homology with the desired mutation is transformed into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Silencing at the Cg2g000710 transcript (RNA) level can be effected using the below exemplary platforms, which are well known in the art. For example, Killiny ei atl www(dot)doi(dot)org/10(dot)1080/15592324(dot)2022(dot)2106079 reported gene silencing in the citrus genome. Li et al. reported on microRNA silencing in the citrus geneome www(dot)doi(dot)org/10(dot)1007/s00299-021-02775-5; another report of a vector for gene silencing in citrus can be found in 10.1007/s40858-020-00357-6, each of which is incorporated by reference in its entirety.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene (Cg2g000710). In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects-see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433, and Diallo et al, Oligonucleotides, Oct. 1, 2003, 13(5): 381-392. doi:10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the Cg2g000710 gene (or its ortholog) mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912(dot)html).

Second, potential target sites are compared to an appropriate genomic database (e.g., human, mouse, rat etc.) using any sequence alignment software, such as the BLAST software available from the NCBI server (www(dot)ncbi(dot)nlm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Constructs useful in the methods according to the present invention may be constructed using recombinant DNA technology well known to persons skilled in the art. The coding sequence constructs may be inserted into vectors, which may be commercially available, suitable for transforming into plants and suitable for expression of the gene of interest in the transformed cells. The genetic construct can be an expression vector wherein the nucleic acid sequence is operably linked to one or more regulatory sequences allowing expression in the plant cells.

Plant cells may be transformed stably or transiently with the nucleic acid constructs of the present invention. Transient transformation can be done for instance in the case of genome editing to exclude the nuclease once editing is achieved.

The selection of citrus candidates for genetic modification (e.g., by any of the methods described herein) can be done the use of molecular markers identifying the loss of function mutation such as those capable of identifying, the transposon sequence is as set forth in SEQ ID NO: 37 (Copia-like LTR retrotransposon). These methods typically employ sequencing, PCR or combinations of same, though any other methods for marker identification is contemplated herein. Such methods can be further employed in validating loss of function of the Cg2g000710 gene or ortholog thereof following the genetic modification e.g., genome editing, breeding etc.

Alternatively, the validation is performed using biochemical methods such as described above to monitor the presence of coumarins and FCs.

The present teachings further relate to plant parts which comprise the loss of function mutation.

These parts include, but are not limited to, a grafted plant, ancestors and progeny of the plants and plant parts, including seeds, shoots, stems, roots, rootstock, scion, and plant cells, tissues and organs. The plant may be in any form including suspension cultures, embryos, meristematic regions, callus tissue, leaves, gametophytes, sporophytes, pollen, and microspores.

According to a specific embodiment, the plant part is a fruit.

According to a specific embodiment, the plant part is a cutting.

According to a specific embodiment, the plant part is a seed.

Embodiments described herein also relate to the germplasm of the plants. The germplasm is constituted by all inherited characteristics of an organism.

Once the plants are at hand they can be used in the citrus industry for any practical application.

Thus, according to an aspect of the invention there is provided a method of producing an edible product, comprising processing the fruit of the plant as described herein (comprising the loss of function mutation in the gene or ortholog).

According to a specific embodiment, the product comprise lower percentage of coumarin or FCs as compared to an identical product in which a citrus plant of the same species was used but without the mutation (e.g., at least 20%, 30%, 40%, but more preferably 50%, 60%, 70%, 80%, 90% or completely absence of coumarins and FCs, as determined by a method described herein).

According to a specific embodiment, the product comprises DNA of the citrus plant.

According to a specific embodiment, the product does not comprises DNA of the citrus plant.

examples of such products include those which comprise citrus peel, pulp, puree, juice, cells, fresh fruit, fruit slices, or dehydrated fruit or peel.

Fruit peel—the most important component of citrus peel is hesperidina, a bioflavonoid which helps metabolise liquids in your blood and reduce/eliminate fat. Peel also contains other bioflavonoids, like naringin (which makes products taste bitter), diosmin (which is beneficial to heart health) and neohesperidin (used as a sweetener). Neohesperidin is a powerful sweetener, popular in the food industry for the great advantages it offers in comparison with other sweeteners such as aspartame, sucralose, etc. Peel also contains pectin, a natural fibre which combats stomach problems and keeps your sugar levels stable. It also has a high vitamin content.

Lemon peel combats abdominal swelling by reducing intestinal gases. it is rich in essential oils that can be used for a wide range of purposes in the food, cosmetics and perfume sectors. Peel is typically processed for the use as infusions, citrus peel confits (confectionery sector), citrus chips, rinds and frozen citrus fruits which can be produced in different formats: squares, strips, zest, etc., sweets and candy, jams and marmalades.

Pulp is a part of the fruit that has been separated, either mechanically or manually, from the peel and seeds. In order to be classed as pulp, this part of the fruit must not have undergone any process of concentration, fermentation or dilution.

Pulp is typically used in jams and marmalades, ice cream, smoothies, frozen citrus pulp, sweet pastries and toppings.

Citrus purees relate to the mashed inner part of the fruit. Typically used for Fruit purees such as for children, cocktails, savory dishes and desserts.

Fruit juice used concentrated or 100% natural fruit juice slushes, a replacement for soft drinks, ice lollies, sweet or savoury dishes. Other products include the fibre obtained after the juice has been sieved and decanted, Comminute: a very aromatic puree obtained by crushing the whole fruit, Dehydrated fruit, cross cuts of whole fruits and fresh fruit.

Any of the above can be branded with identification of their lower coumarin/FCs content and therefore prevent citrus/drug interactions. Such interactions are common to various drugs such as statins that prevent high cholesterol, drugs that treat high blood pressure, organ-transplant rejection drugs, anti-anxiety drugs, corticosteroids that treat Crohn's disease or ulcerative colitis, drugs that treat abnormal heart rhythms and antihistamines that treat allergic reactions (www(dot)fda(dot)gov/consumers/consumer-updates/grapefruit-juice-and-some-drugs-dont-mix).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Methods

Plant Material and Chemicals Trees of ‘Ora’ mandarin (Citrus reticulata Blanco), ‘Hudson’ grapefruit (C. paradisi) ‘Pazit’ mandarin and ‘Chandler’ pumelo (C. grandis), and plants of F1 hybrid progenies were grown in the orchard and in a net-house at the Volcani Center, Rishon LeZion, Israel. Young leaves, roots and fruitlets were collected, frozen in liquid nitrogen and kept at −80° C. until further analysis.

Scopoletin, umbelliferon, demethylsuberosin, marmesin, psoralen, bergaptol, bergamottin and 6,7-dihydroxybergamottin analytical standards (purity >98%) were purchased from Sigma-Aldrich (Steinem, Germany), Fluka (Buchs, Switzerland) and Cayman (Ann Arbor, MI, USA).

### SNP Analysis to Identify Hudson X Ora F1 Hybrid Plants

For validation of mapping populations, a subset of SNPs was chosen for identification of progenies of the crosses {grapefruit ♀ (cv. Hudson) X mandarin ♂ (cv. Ora)} and {pumelo ♀ (cv. Chandler) X mandarin ♂ (cv. Pazit)} based on a work done on a proprietary germplasm using known SNPs for citrus 41. SNP type assays were designed by the D3 site to be compatible with the Fluidigm genotyping platform (www(dot)d3.fluidigm(dot)com/, (not shown). Crude DNA from young leaves was made using the protocol that was published by Xin et al, 2003⁴² with minor modifications. The DNA was diluted at 1:20 and used together with the SNP type assay on the EP1 genotyping platform with 24×192 arrays. Standard protocols and analysis were used besides adding more negative controls on every array (www(dot)fluidigm(dot)com). Results were checked manually and compared to the parents of the cross. Only correct progenies were selected. Correct progenies are the ones that contain both maternal and paternal DNA.

### Coumarin and Furanocoumarin Extraction

Coumarins and FCs were extracted from young leaves, roots and peel. For each sample, 200 mg of tissue were ground in liquid nitrogen and added to 600 ul ethyl acetate. The extraction was performed by vortex mixing for 5 min at 25° C., followed by centrifugation at 13,000 rpm for 10 min at 25° C. The organic phase was collected and re-extracted with an equivalent volume of double-distilled water. The mixture was then remixed and centrifuged as described above, for two additional times. The organic phase was evaporated in a chemical hood and dissolved in 1 ml of acetonitrile for LC-MS analysis.

### Liquid Chromatography—Mass Spectrometry

LC-MS analyses were conducted using a UPLC-Triple Quadrupole-MS (Waters Xevo TQ MS). Samples were filtered through a 0.22-μm Millex-HV Durapore (PVDF) membrane before injection into the LC-MS apparatus. Separation was performed with a 2.1 m×100 mm i.d., 1.7 μm UPLC BEH C18 column. Chromatographic and MS parameters were as follows: the mobile phase consisted of water and 0.1% formic acid (phase A) and 0.1% formic acid in acetonitrile (phase B). The gradient program for bergamottin, 6,7-dihydroxybergamottin, bergaptol, psoralen, umbelliferon and DMS was as follows 95% A over 0.5 min, 95% to 5% A over 8 min, held at 5% A for 3 min, then back to the initial conditions (95% A) for 1 min and kept under the initial conditions for 3 min. The flow rate was 0.3 ml·min−1 and the column temperature was kept at 35° C. The analyses were performed with the ESI source used in positive-ion mode, with a capillary voltage of 3.2 kV, a cone voltage of 30 V, a desolvation temperature of 350° C., a desolvation gas flow of 650 L/h and a source temperature of 140° C. Gradient program for scopoletin and marmesin was as follows 95% A over 0.5 min, 95% to 60% A over 14.5 min, 60% A to 5% A over one minute, held at 5% A for 3 min, then back to the initial conditions (95% A) for 1 min and kept under the initial conditions for 3 min. The flow rate was 0.3 ml·min−1 and the column temperature was kept at 35° C. The analyses were performed with the ESI source used in positive-ion mode, with a capillary voltage of 3.2 kV, a cone voltage of 30 V, a desolvation temperature of 350° C., a desolvation gas flow of 650 L/h and a source temperature of 140° C. Quantitation was performed with MRM acquisition by monitoring the following transitions 339/147, 339/203 (RT=8.33) for bergamottin; 373/153, 373/203 (RT=5.39) for 6,7-DHB; 203/91, 202/146 (RT=4.37) for bergaptol; 187/115, 187/131 (RT=4.85) for psoralen 163/91, 163/107 (RT=3.51) for umbelliferon; 193/133, 193/178 (RT=6.60) for scopoletin; 231/91, 231/175 (RT=5.91) for DMS; 247/91, 247/229 (RT=9.76) for marmesin; 165/119, 165/147 (RT=3.30) for coumaric acid; 195/145, 195/177 (RT=9.76) for ferulic acid. Acquisition of LC-MS data was performed under MassLynx V4.1 software (Waters). Quantification was done using calibration curves.

RNA Isolation, cDNA Library Construction and RNA-Seq.

For RNA Seq, total RNA was extracted from citrus young leaves using Plant Total RNA Purification Kit (Norgen Biotek Corp., ON, Canada).

RNA Library preparation and sequencing were performed at Macrogen Europe, Maastricht, The Netherlands. Paired-end RNA-Seq libraries were prepared using Illumina and Trueseq RNA protocols. Raw reads were subjected to a filtering and cleaning procedure. First the Trimmomatic tool⁴³ was used to remove Illumina adapters from the reads. Next, the FASTX Toolkit (www(dot)hannonlab.cshl.edu/fastx_toolkit/index(dot)html, version 0.0.13.2) was used to trim the read-end nucleotides with quality scores <30 (FASTQ Quality Trimmer), and to remove reads with less than 70% base pairs with a quality score ≤30 (FASTQ Quality Filter). Clean-reads were aligned to the Citrus maxima v1.0 (C. grandis) reference genome extracted from the Citrus genome database (www(dot)citrusgenomedb(dot)org/) using STAR software (v2.7.1a)⁴⁴ with an average mapping rate of 91.15%. Gene abundance was estimated using Cufflinks workflow (v. 2.2)⁴⁵ combined with gene annotations from Citrus genome database.

Cg2g000710 Enzyme Activity Assay in N. benthamiana Leaves

The coding sequence of the Cg2g000710 grapefruit orthologue was amplified from cDNA of ‘Hudson’ grapefruit leaves with primers Fw 5′ CCACACTAGTAACATGGCACCAACAAATGGC 3′ (SEQ ID NO: 5), and Rev 5′ GAAACCAGATCGTTGGTCAAAAGTCACCTATCTTTG 3′ (SEQ ID NO: 6), using Phusion High-Fidelity DNA Polymerase (Thermo Fisher Scientific, Waltham, MA, USA). The amplified fragment was cloned into the binary vector pH2GW7 (Ghent University, Ghent, Belgium) at the SpeI and BstXI restriction sites downstream of a CAMV-35S promoter. The resulting pH2GW7-Cg2g000710 construct was used to transform Agrobacterium tumefaciens EHA-105 cells. The same was done for R. graveolens C2′H/F6′H coding sequence to be used as a positive control for activity¹⁵, whereas expression of the mCherry red fluorescent plasmid was used as negative control²⁷.

Agrobacterium harboring plasmids for expression were co-infiltrated with agrobacterium harboring P19 silencing suppressor into 4 weeks-old N. benthamiana leaves. After 48 hours, substrates (coumaric acid; CA or ferulic acid; FA) were infiltrated into the same leaves as described previously⁴⁶, and following an additional 72 hours leaves were harvested and furanocoumarin products were extracted for LC-MS analysis.

### DNA Isolation and Diagnostic PCR Analysis

Genomic DNA was extracted from young citrus leaves using GenElute Plant Genomic DNA Miniprep Kit (Sigma-Aldrich, Steinem, Germany). PCR for Cg2g000710 and orthologues allele distribution analysis was preformed using three primer pairs: (1) set of primers designed to produce a product only in the presence of a 655 base insertion within the reading frame {Insertion allele; Fw 5′ ATGGCACCAACAAATGGCTCTACCCTC 3′ (SEQ ID NO: 7), and Rev 5′ GCTTTTCCTGTGGCTTTTCCCGTGAATGT 3′ (SEQ ID NO: 8)}, (2) set of primers designed to produce a short product (743 bp) representing an intact allele specifically in our F1 populations {Intact allele; Fw (same as Rev 5′ above), and GTACAGTACACAATCTTAGGTATCATGTTTGG 3′ (SEQ ID NO: 9)), and (3) set of primers designed to produce a short product (459 bp) representing an intact allele in various species of the citrus genetic collection {Intact allele; Fw (same as above), and Rev 5′ TAGGCTGAGAAAGTCTTTCCACTCCAAAGCCT 3′ (SEQ ID NO: 10)}.

### Phylogenetic Analysis for Previously Reported and Putative 2OGDs

All sequences for phylogenetic analysis were obtained from the National Center for Biotechnology Information database (NCBI; www(dot)ncbi(dot)nlm(dot)nih(dot)gov/), The Citrus Genome Database (www(dot)citrusgenomedb(dot)org/) and Phytozome (www(dot)phytozome-next(dot)jgi(dot)doe(dot)gov/). Phylogenetic tree construction was performed on previously characterized 2OGDs and C. grandis encoded proteins (Citrus maxima, v1.0 HZAU) based on homology, using the MUSCLE algorithm for alignment, PhyML for phylogeny and TreeDyn viewer (www(dot)phylogeny(dot)fr/). Homology is a reliable tool to identify members of the 2OGD family (or any other gene family).

### Quantitative Reverse Transcription PCR Analysis

For q-RT PCR purposes, total RNA was extracted from fruitlets and roots according to the CTAB protocol⁴⁷. RNA concentrations were determined using NanoPhotometer NP80 (Implen GmbH, München, Germany). RNA samples were than treated for removal of genomic DNA contamination and served as templates for cDNA synthesis using All-In-One 5×RT MasterMix (abm, BC, Canada). Q-RT PCR was performed in three replicates using PowerTrack SYBR Green Master Mix on a StepOnePlus Real-time PCR detection system (Thermo Fisher Scientific, Waltham, MA, USA). Gene expression in each sample was normalized to FBOX (unigene CAS-PT-306416) as the internal reference gene as described for citrus gene expression using FBOX gene specific primers⁴⁸. All primers used for qRT-PCR are listed in Table 4.

TABLE 4

| | Orien- | |
|---|---|---|
| Gene | tation | Sequence 5′-3′ |
| Cg2g000700 | Fw | CTTCTTGTGATACTAAGTCCAAGCA |
| | | (SEQ ID NO: 11) |
| Cg2g000700 | Rev | CAGGCTTTTGAGTCCCATTTCG |
| | | (SEQ ID NO: 12) |
| Cg2g000710 | Fw | CTTGGAAGAAAGATTCAGTGAGAAG |
| | | (SEQ ID NO: 13) |
| Cg2g000710 | Rev | GACTTCAGGACTCTCCCAGT |
| | | (SEQ ID NO: 14) |
| Cg2g000720 | Fw | CCTTCAGAGCAAAGTCCAAAC |
| | | (SEQ ID NO: 15) |
| Cg2g000720 | Rev | GTATGACAAAATCAGCAATATCCC |
| | | (SEQ ID NO: 16) |
| FBOX | Fw | TTGGAAACTCTTTCGCCACT |
| | | (SEQ ID NO: 17) |
| FBOX | Rev | CAGCAACAAAATACCCGTCT |
| | | (SEQ ID NO: 18) |

### Example 1

### Segregation of Furanocoumarin/Coumarin Biosynthesis in Citrus Cross-Species F1 Populations

In order to provide a genetic basis for an in-depth study of the enzymatic steps and corresponding genes of FC biosynthesis in citrus, the present inventors generated two F1 populations: (1) a cross between a mandarin ♀ (cv. Pazit) and a pumelo ♂ (cv. Chandler); (2) a cross between a mandarin ♀ (cv. Ora) and a grapefruit ♂ (cv. Hudson). Parental lines and F1 progeny (confirmed by SNP analysis as described in the materials and methods) were subject to metabolomic analyses to determine the content of FCs, as well as the coumarin FC-precursor umbelliferon (UM), in young leaves.

TABLE 5
Pazit X Chandler (C.reticulata X C.grandis) and Ora X
Hudson (C.reticulata X C.paradisi) populations F1 progeny
varification with Fluidigm

| | | | Fluidigm | | | | | | |
|---|---|---|---|---|---|---|---|---|---|
| | | Cross | test | No. of | Codes of | No. of | | | |
| | | preformed | preformed | offsprings | tested | true | DNA | SNP pl | |
| Female | Male | at | at | tested | plants | offsprings | plates | No. | Summary |
| Ora | Hudson | 03.2015 | 08.2016 | 140 | Cit-OHG- | 60 | 16-17 | 10 | Array 4 |
| | | | | | 1-136 | | | | |
| | | | | | Cit- | | | | |
| | | | | | OHB- | | | | |
| | | | | | 1-4 | | | | |
| Ora | Hudson | 03.2020 | 06.2021 | 465 | OH | 403 | 50-51, | 27 | Array |
| | | | | | 300-764 | | 52-53 | | 19-21 |
| Pazit | Chandle | 03.2016 | 02.2018 | 87 | Cit_PC_ | 76 | 38-39 | 22 | Array 14 |
| | | | | | 1-87 | | | | |

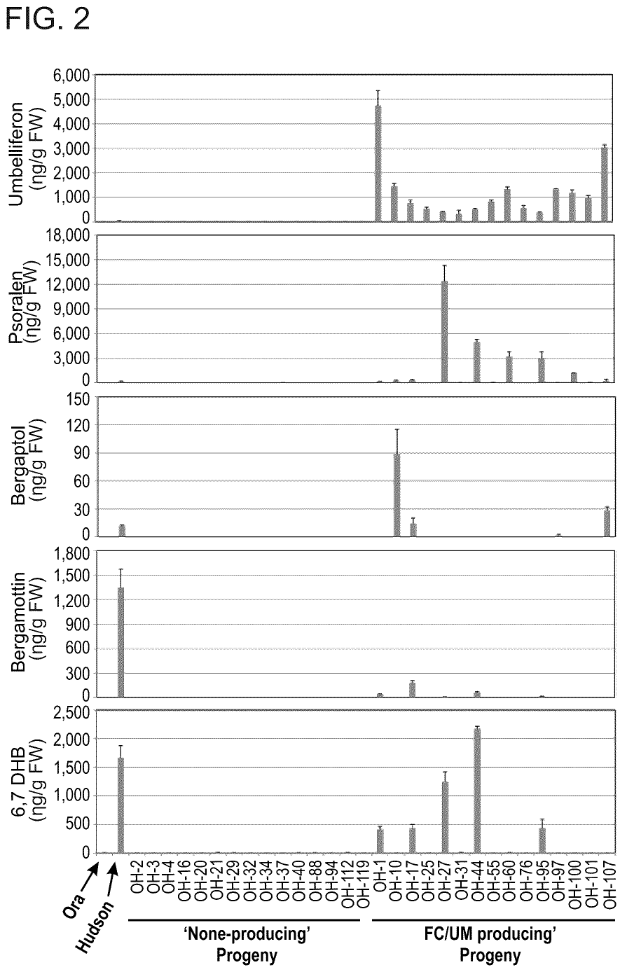

The level of the FCs/UM in leaves of the parental lines were found to be in-line with the levels previously reported for various mandarin, pumelo and grapefruit varieties²⁶; ‘Pazit’ and ‘Ora’ mandarin leaves were found to lack FCs/UM, while ‘Chandler’ pumelo and ‘Hudson’ grapefruit contained high levels of the FCs psoralen and 6,7-DHB, as well as low but significant levels of the FC precursor UM (FIG. 2).

Significantly, the two F1 populations displayed distinctively different inheritance patterns for FC accumulation. While the mandarin ♀ (cv. Pazit) X pumelo ♂ (cv. Chandler) F1 progeny (30 plants) displayed relatively high uniform accumulation level of FCs (not shown), the mandarin ♀ (cv. Ora) X grapefruit ♂ (cv. Hudson) F1 progeny (142 plants) segregated for FC/UM biosynthesis at a close to 50/50 ratio; 76 of the F1 plants had undetectable or negligible FC/UM levels (i.e. ‘Non-producing’ progeny) and 66 of the F1 plants produced significant levels of FCs and/or UM (i.e. ‘FC/UM producing’ progeny) (FIG. 2). Thus, the FC/UM biosynthesis inheritance pattern, when considering both populations, is consistent with inheritance of functional/non-functional alleles of a single gene involved in umbelliferon biosynthesis; according to this model ‘Pazit’ and ‘Ora’ mandarins are homozygous for a non-functional allele while ‘Hudson’ grapefruit is heterozygous for a functional allele and ‘Chandler’ pumelo is homozygous for a functional allele.

### Example 2

### Mining a Candidate Gene for Umbelliferon Biosynthesis in the Pumelo Genome

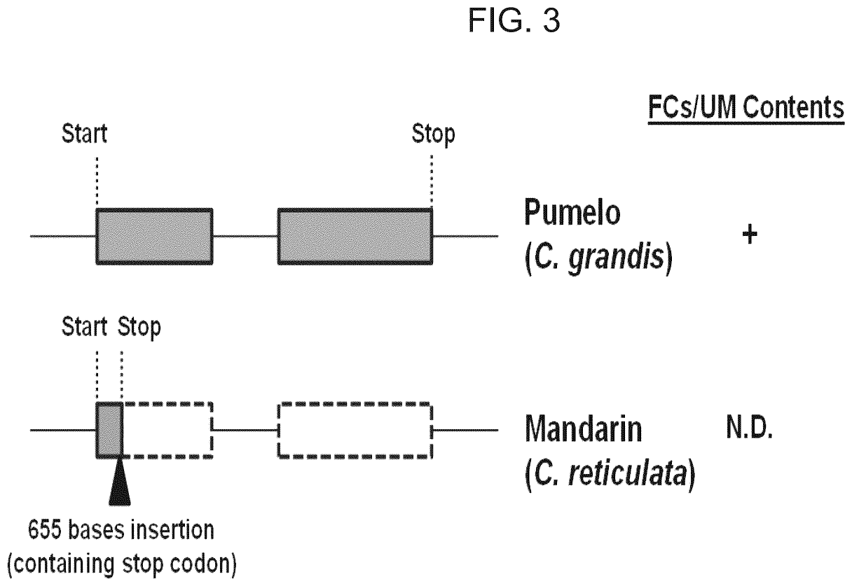

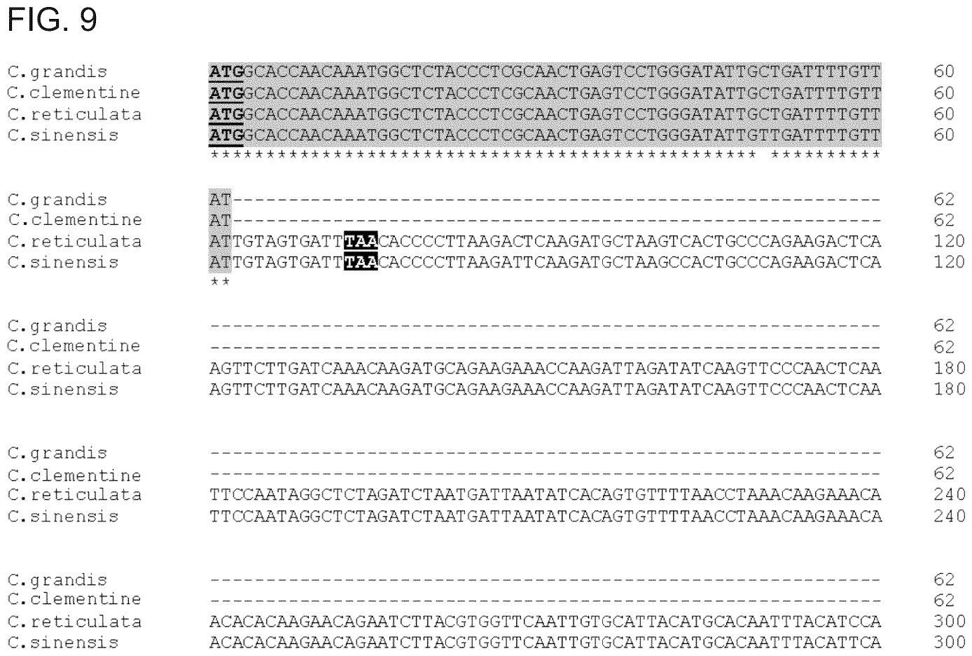

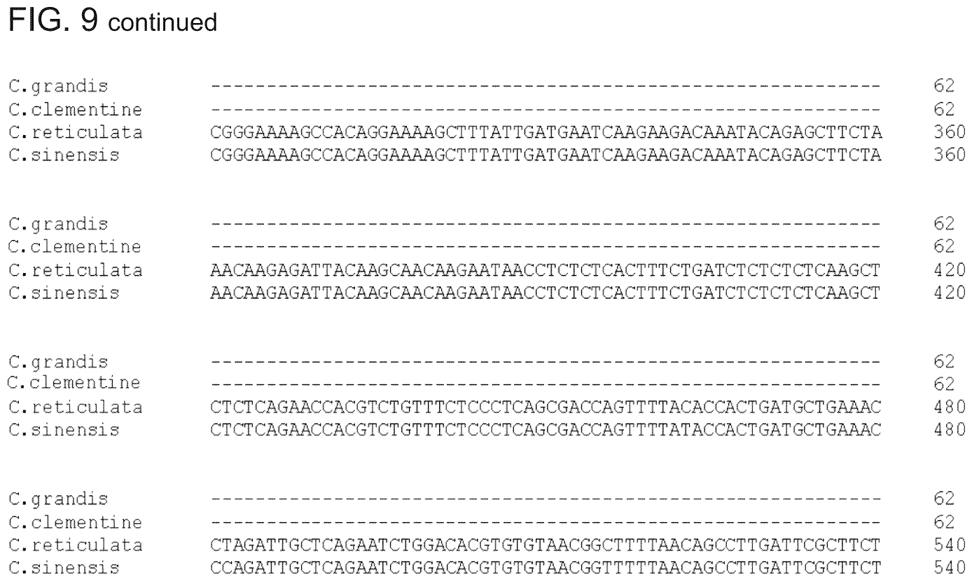

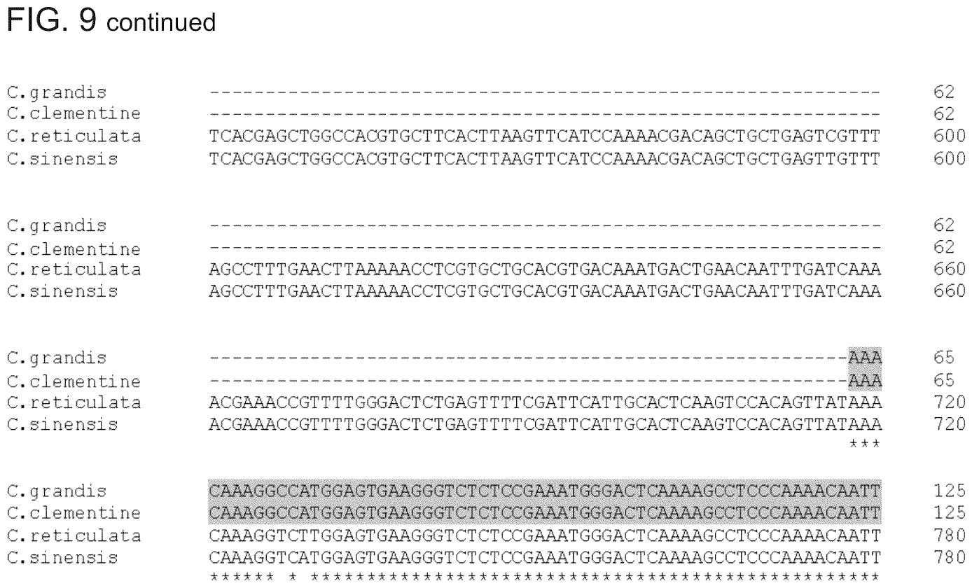

Based on the working hypothesis, that inheritance of a single gene involved in umbelliferon biosynthesis can explain the observed segregation pattern of FC/UM biosynthesis in the mandarin ♀ (cv. Ora) X grapefruit ♂ (cv. Hudson) F1 population, the present inventors commenced a candidate gene search by analyzing young-leaf transcriptomes of the parental lines (assembled from RNA-seq data as described in the materials and methods) and using pumelo (C. grandis) (an ancestral source for FC biosynthesis) as a reference genome (it is noted that a grapefruit reference genome was not available at the time of this research). 2OGDs of the p-coumaroyl CoA 2′-hydroxylase (C2′H)/feruloyl-CoA 6′-hydroxylase (F6′H) subgroup catalyze umbelliferon/scopoletin biosynthesis, respectively, in common rue (Ruta graveolens), sweet potato (Ipomoea batatas), parsnip (Pastinaca. sativa) and Peucedanum praeruptorum^{15,19-21}. The integrity and expression of the 2OGD family genes were investigated in the young-leaf transcriptome of ‘Hudson’ grapefruit (which contains FCs/UM in fruit/leaves) compared to the corresponding genes in the young-leaf transcriptome of ‘Ora’ mandarin (which lacks FCs/UM in fruit/leaves). The screen for a candidate gene yielded a putative 2OGD that was expressed and appeared to have an intact reading frame in the ‘Hudson’ grapefruit leaf transcriptome and in the pumelo genome (Cg2g000710), but was absent from the ‘Ora’ mandarin transcriptome, and has a disrupted reading frame in the mandarin genome due to a 655 base insertion, just 60 bases (20 AA) downstream of the first methionine codon (MSYJ070210) (FIG. 3). Furthermore, analysis of the clementine and sweet orange genomes demonstrate an identical 655 base insertion sequence interrupting the orthologous 2OGD reading frame (Ciclev10017914m and Cs9g02910, respectively), resulting in a premature stop codon (FIG. 9 where the sequences are SEQ ID Nos: 29-32). Thus, the young leaf transcriptome results, supported by the available genomic data (pumelo, mandarin, clementine and orange), supports the genetic model that was suggested based on phenotyping of the population.

### Example 3

### Cg2g000710 Encodes a Dual C2′H/F6′H Enzyme Catalyzing Biosynthesis of the Coumarins Umbelliferon and Scopoletin

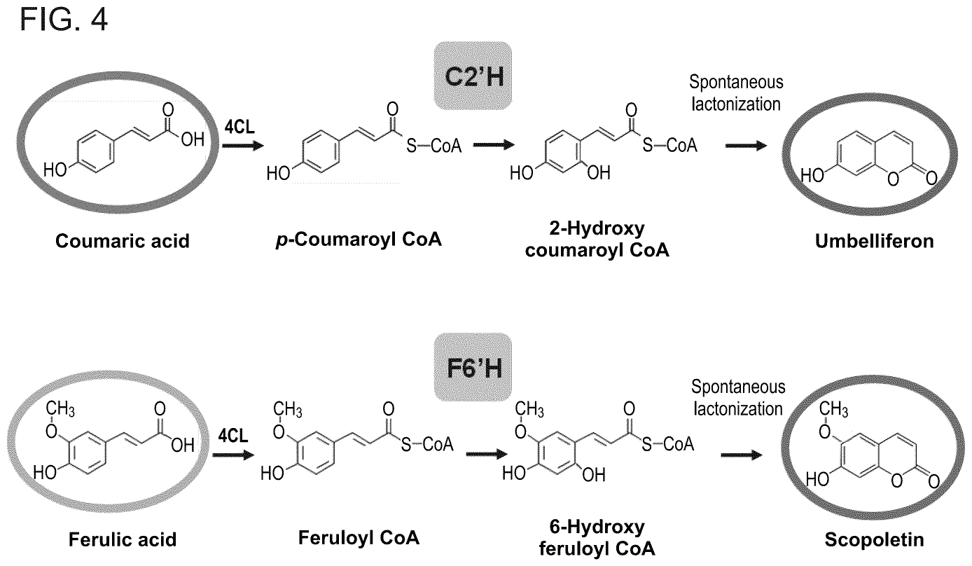

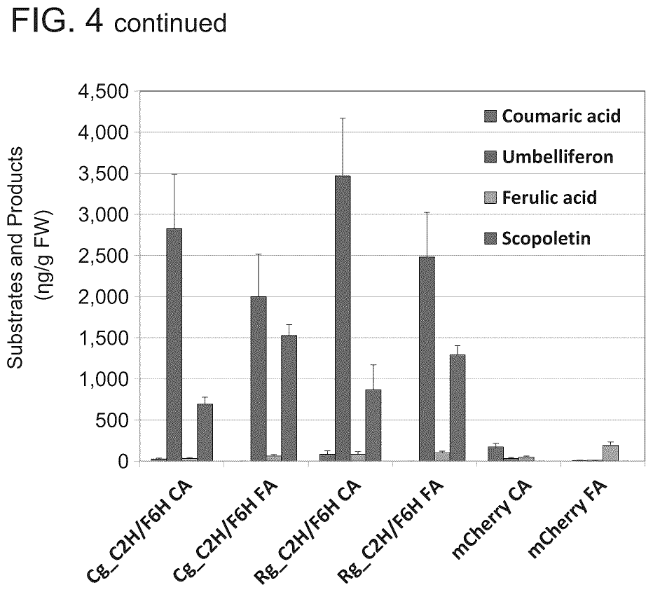

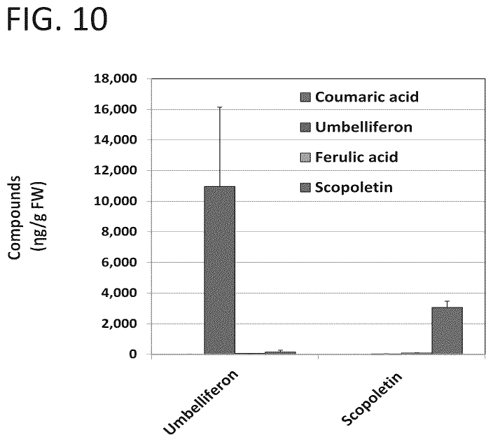

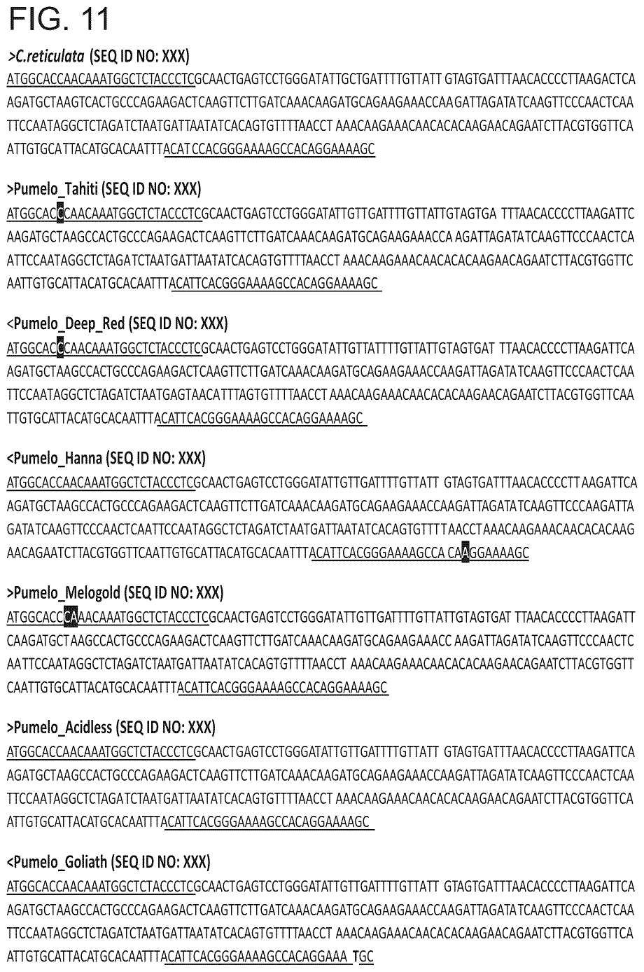

The reading frame of Cg2g000710 was used to analyze the encoded enzyme activity in-planta by transient expression in leaves of Nicotiana benthamiana (FIG. 4). Agrobacterium lines harboring constructs for expression of the Cg2g000710 reading frame SEQ ID NO: 26, a positive control for C2′H/F6′H activity (reading frame of the R. graveolens C2′H/F6′H) 15 3 replicates for each of the treatments or a negative control (reading frame of the red fluorescent protein mCherry) 27 without enzymatic activity-negative control, were infiltrated into N. benthamiana leaves. The substrate precursors coumaric acid or ferulic acid (which convert in-planta to p-coumaroyl CoA and feruloyl CoA, respectively, by 4CL15) were subsequently infiltrated into these leaves after 48 hours. Finally, leaves were harvested for metabolite extraction and analysis after an additional 72 hours. While accumulation of umbelliferon or scopoletin was negligible in the negative control (mCherry red fluorescent protein) infiltrated leaves, expression of either R. graveolens C2′H/F6′H (positive control) or the reading frame of Cg2g000710 resulted in dramatic accumulation of the coumarin products umbelliferon and scopoletin (66 and 385 fold higher than the control plasmid, respectively), demonstrating that Cg2g000710 encodes a C2′H/F6′H enzyme (FIG. 4), which was designated as CgC2′H/F6′H. Interestingly, infiltration of either substrate precursor (coumaric acid or ferulic acid) resulted in both coumarin products, however with an advantage to umbelliferon. Since the present inventors ruled out conversion of scopoletin to umbelliferon in the plant tissue (or vice-versa) (FIG. 10) it appears that part of the coumaric acid infiltrated is converted in the plant to ferulic acid and vice-versa.

### Example 4

### Integrity of Cg2g000710 and its Orthologues are in Direct Correlation with the Capacity to Synthesize FCs/UM in Leaves of the F1 Populations

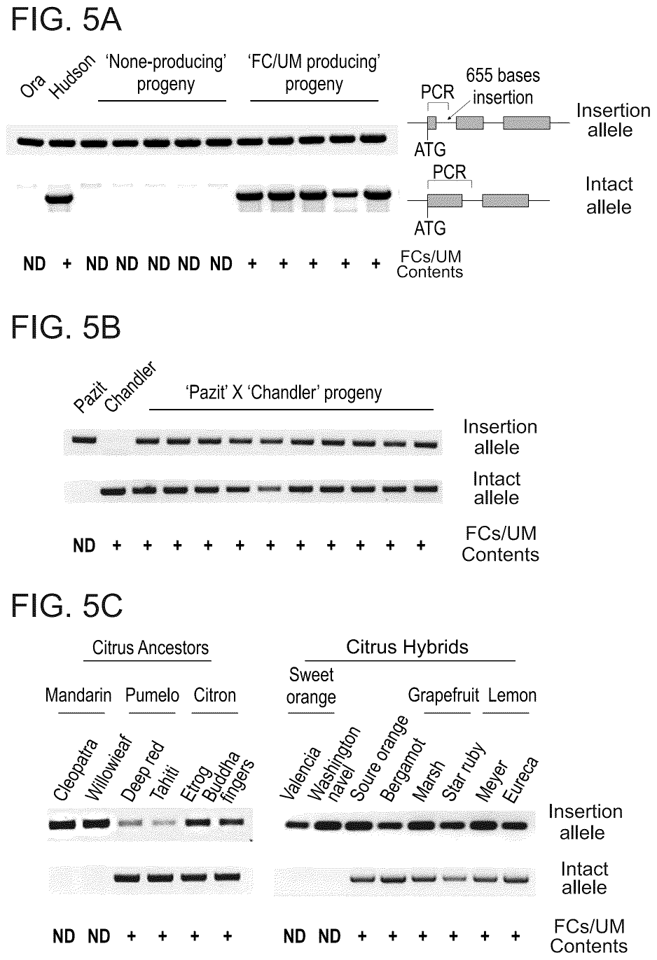

In order to study the correlation between integrity of the Cg2g000710 gene (characterized as a CgC2′H/F6′H) orthologues and the capacity to synthesize FCs/UM in leaves of the F1 populations, the parental lines and F1 plants were scored for FC/UM levels and for presence/absence of the 655 base reading-frame disrupting insertion sequence. The results demonstrate a consistent correlation between FC/UM accumulation in leaves and the presence of at least one intact Cg2g000710-like allele (FIGS. 5A and B). The parental line ‘Ora’ mandarin, which does not produce FCs/UM, is homozygous for the 655 base disrupting insertion (MSYJ070210) and therefore lacks an intact allele, while the second parental line ‘Hudson’ grapefruit, which accumulates FCs, has one intact Cg2g000710-orthologue allele and one disrupted allele (FIG. 5A). Consequently, the F1 progeny of the mandarin ♀ (cv. Ora) X grapefruit ♂ (cv. Hudson) cross segregate approximately 50/50 for FC/UM accumulation in leaves in direct correlation with the presence of an intact CgC2′H/F6′H-orthologue allele (FIG. 5A).

Analysis of the second F1 population {pumelo ♀ (cv. Chandler) X mandarin ♂ (cv. Pazit)} and the respective parental lines provided further support for the correlation between integrity of the characterized CgC2′H/F6′HI gene and the capacity to synthesize FCs/UM in leaves. While ‘Pazit’ mandarin lacks FCs/UM and correlatively lacks an intact CgC2′H/F6′H-orthologue allele (i.e., homozygous for MSYJ070210 containing the 655 base disrupting insertion), ‘Chandler’ pumelo displayed high levels of FCs in leaves (FIG. 5B) and is homozygous for an intact Cg2g000710 allele (FIG. 5B). The F1 progeny are all heterozygous (i.e., contain one intact allele and one disrupted allele) in correlation with high levels of FCs in leaves.

The present inventors further investigated the integrity of the characterized Cg2g000710 gene and its orthologues in correlation to FC/UM accumulation in other citrus species/varieties (FIG. 5C). Analyzed mandarin varieties (‘Cleopatra’, ‘Willowleaf’) and sweet orange varieties (‘Valencia’, ‘Washington navel’) were found to be homozygous for the 655 base insertion sequence and in correlation lack FCs/UM (FIG. 5C).

Interestingly, all other citrus varieties tested, including citrons, grapefruits, pumeloes, sour orange, Bergamot and lemons, are heterozygotes and contain one intact allele and one disrupted allele (FIG. 5C). The only variety found to be homozygous for an intact allele of Cg2g000710 was ‘Chandler’ pumelo, as mentioned above (FIG. 5B). In order to examine allele distribution within additional pumelo varieties, the present inventors assayed for presence of the 655 base disrupting insertion (FIG. 5B, SEQ ID NO: 37).

### Example 5

### The 655 Base Insertion Sequence is Likely a Solo-LTR Element Originating from a Copia-Like LTR Retrotransposon

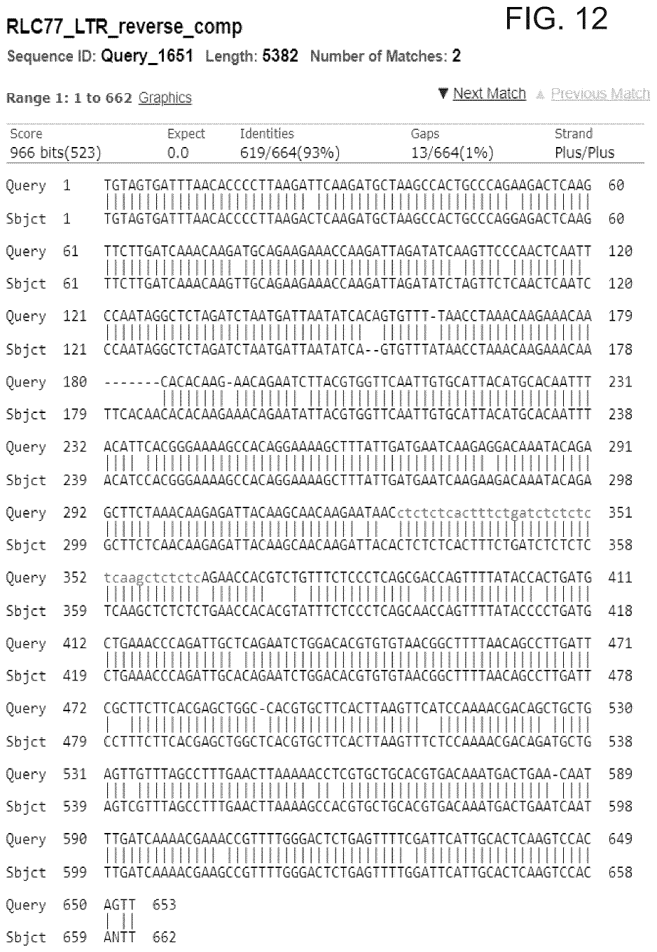

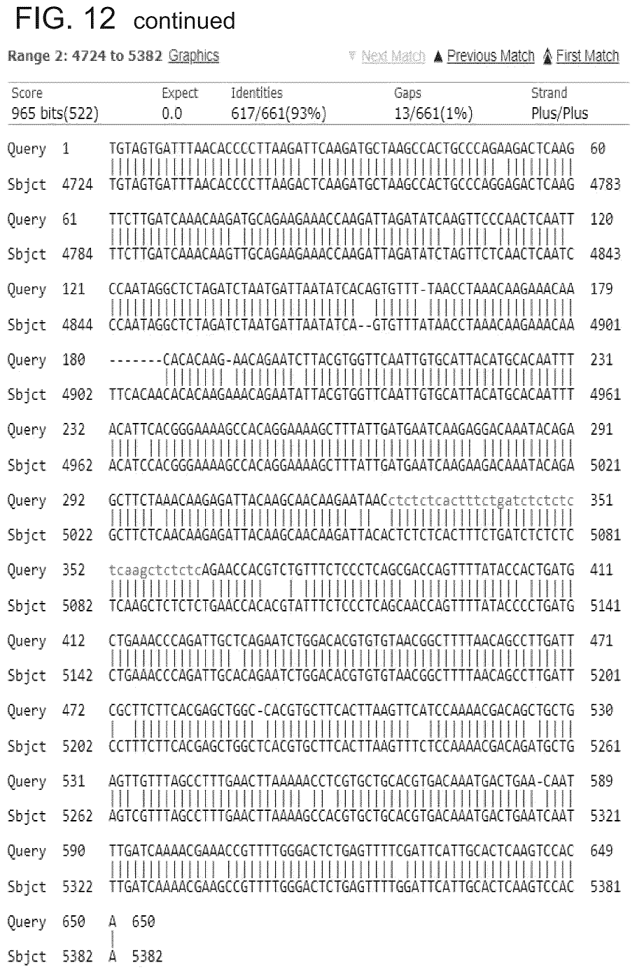

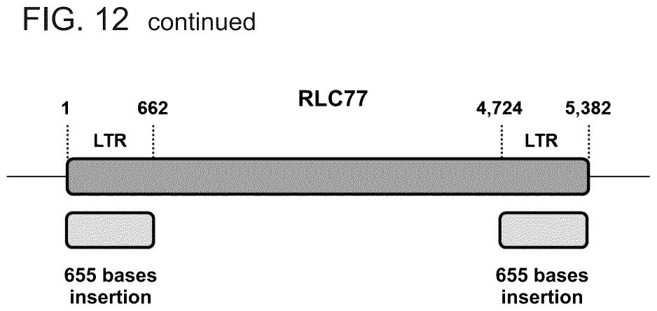

The origin of the 655 base insertion sequence was investigated by bioinformatics tools. BLAST analysis demonstrated prevalence of homologs of the 655 base insertion sequence in all citrus genomes as well as in Atalantia buxifolia (a citrus relative outside of the genus Citrus) (not shown), but homologs were not found outside of the Rutaceae family. The 655 base sequence homologs occur throughout the citrus genomes, in some cases occurring as single elements and in other cases as a pair of elements in relative proximity to each other (not shown). Inspection of the latter type showed that they constitute part of a 5,376 base LTR-retrotransposon-like sequence consisting of pol/gag-like genes flanked by the 655 base sequences occurring as direct repeats (FIG. 12). These retrotransposon-like sequences are ~93% identical to Copia-like LTR retrotransposon family RLC77 from Citrus^{28,29} (FIG. 12). Thus, the 655 base insertion appears to be a solo-LTR^{28,30} derived from a Copia-like retrotransposon.

### Example 6

### Cg2g000710 (Encoding CgC2′H/F6′H) is Part of a Multi-2OGD Gene Locus

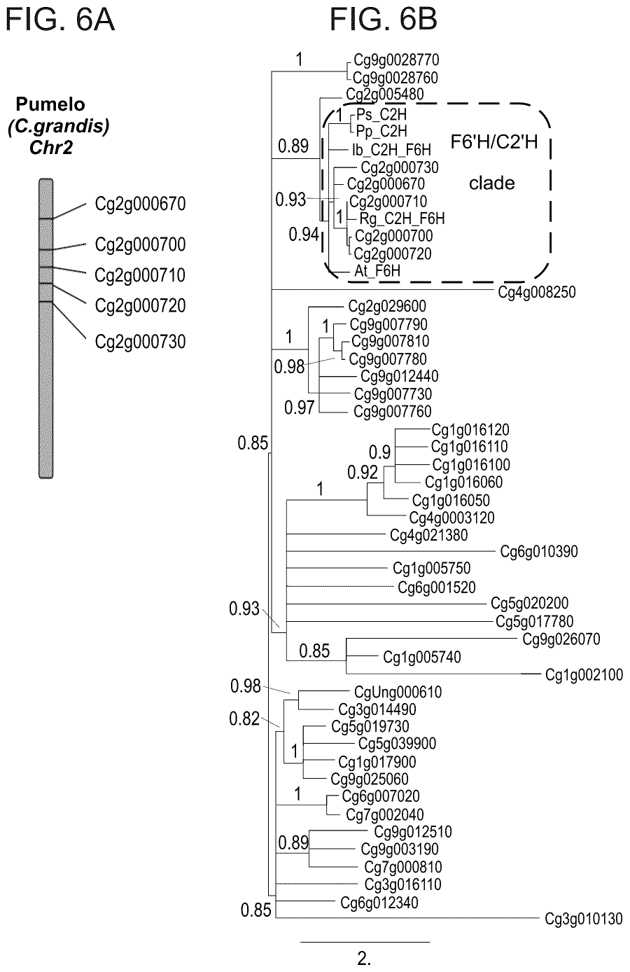

At the genome level, Cg2g000710 (encoding CgC2′H/F6′H) was found to occur within a locus flanked by two additional, almost identical, putative 2OGD encoding genes (Cg2g000700 and Cg2g000720; FIG. 6A). Furthermore, the extended locus includes two additional, yet less similar, putative 2OGD encoding genes (Cg2g000670 and Cg2g000730) (FIG. 6A).

TABLE 6

| | | Cg2g000670 | Cg2g000700 | Cg2g000710 | Cg2g000720 | Cg2g000730 |
|---|---|---|---|---|---|---|
| 1 | Cg2g000670 | 100 | 71.47 | 71.75 | 70.64 | 72.18 |
| 2 | Cg2g000700 | 71.47 | 100 | 93.94 | 95.04 | 60.84 |
| 3 | Cg2g000710 | 71.75 | 93.94 | 100 | 94.49 | 61.19 |
| 4 | Cg2g000720 | 70.64 | 95.04 | 94.49 | 100 | 59.79 |
| 5 | Cg2g000730 | 72.18 | 60.84 | 61.19 | 59.79 | 100 |

This multi-2OGD gene locus is conserved in all sequenced Citrus genomes (mandarin—C. reticulata; pumelo—C. grandis; citron—C. medica; sweet orange—C. sinensis; clementine—C. clementina; lemon—C. limon; kumquat—C. hindsii; trifoliate orange—C. trifoliata and papeda—C. ichangensis) (Not shown but at least about 85% identity, e.g., above 90%, 95% as determined by Blast) (www(dot)citrusgenomedb(dot)org/). At the phylogenetic level, the 5 encoded protein sequences form a distinct sub-clade of 2OGDs together with previously characterized C2′H, F6′H, or dual C2′H/F6′H enzyme sequences from several species (FIG. 6B). However, the 3 highly similar encoded protein sequences, Cg2g000700, Cg2g000710 and Cg2g000720, form a separate subclade together with R. graveolens C2′H/F6′H (FIG. 6B).

### Example 7

### Genes of the 2OGD Locus Specialize in Tissue-Specific Expression

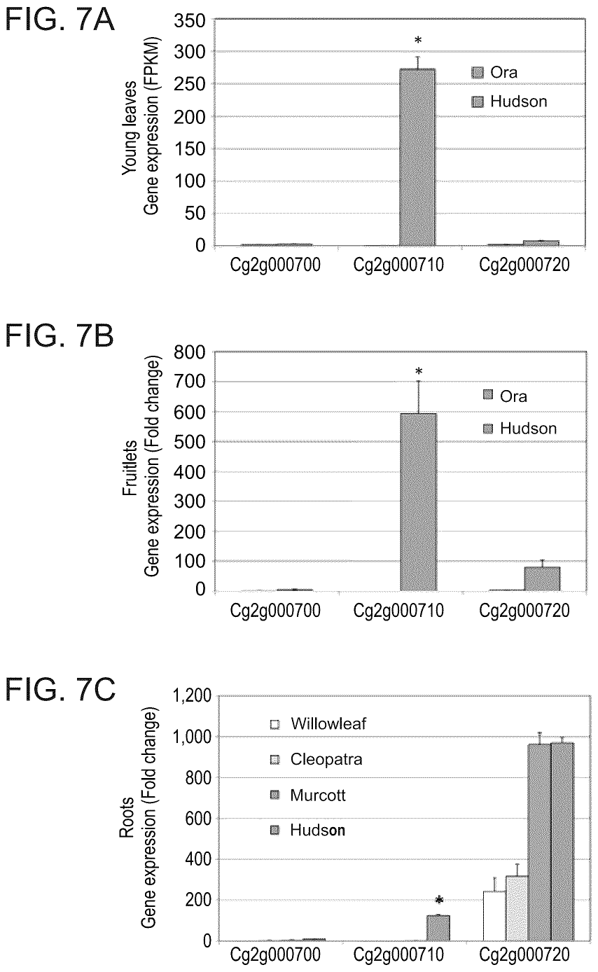

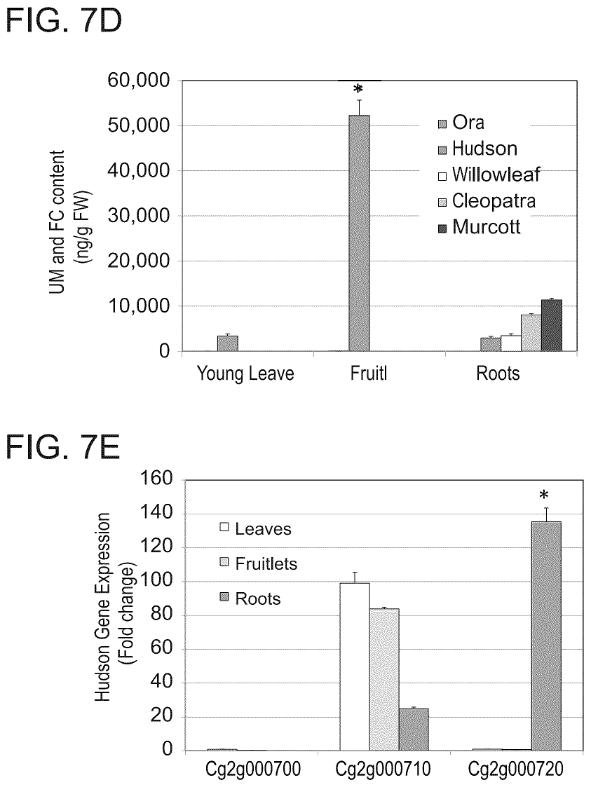

RNA-Seq data was used to study expression levels of the 2OGD locus genes in ‘Hudson’ grapefruit and ‘Ora’ mandarin young leaves (FIG. 7A, Table 7).

TABLE 7
Gene expression levels (FPKM) in young leaves of
‘Ora’ and ‘Hudson’ parental lines

| | *Length | | | | Hudson |
|---|---|---|---|---|---|
| | (AA) | Ora | Ora se | Hudson | se |
| Cg2g000670 | 362 | 2.85 | 0.82 | 0.36 | 0.16 |
| Cg2g000700 | 364 | 1.05 | 0.30 | 2.76 | 0.22 |
| Cg2g000710 | 364 | 0.18 | 0.06 | 272.01 | 19.19 |
| Cg2g000720 | 364 | 1.82 | 0.31 | 6.83 | 0.89 |
| Cg2g000730 | 312 | 0.00 | 0.00 | 0.00 | 0.00 |

The Cg2g000710 orthologue was found to be expressed in high levels in ‘Hudson’ grapefruit (which contains an intact allele), while expression of the corresponding gene in Ora mandarin (homozygous for the solo-LTR disruption) was ~1500 fold lower (~272 vs. 0.18 FPKM, respectively). The other four genes of the locus display very low to undetectable expression levels in both Hudson and Ora leaves (FIG. 7A). Thus, expression of the Cg2g000710 orthologue is the single gene in the cluster differentially expressed in ‘Hudson’ leaves compared to ‘Ora’, and it's expression directly correlates with presence of the solo-LTR disrupting insertion and with FCs/UM levels (FIGS. 2, 5A and 7A).

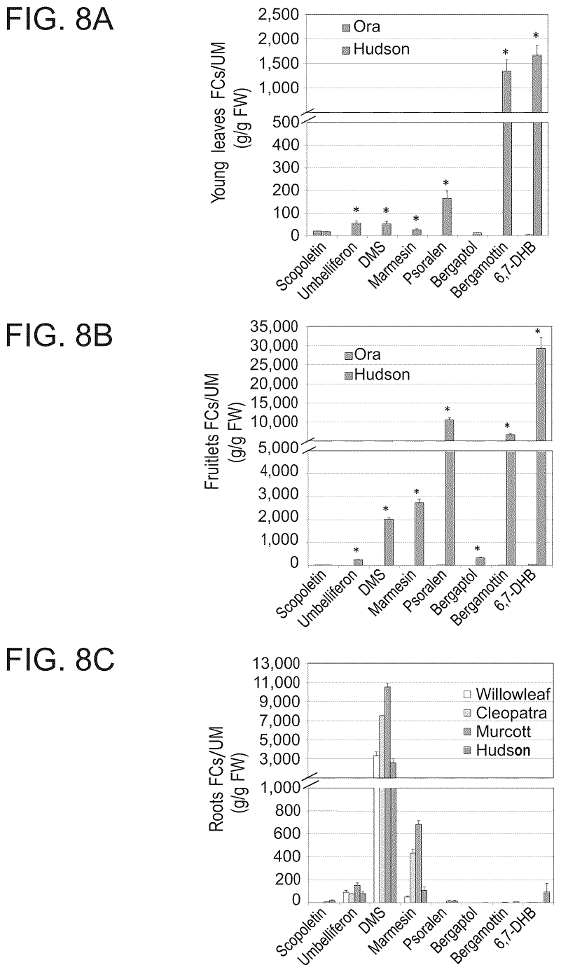

The present inventors further studied relative expression of the 3 highly similar 2OGD locus genes (orthologues of Cg2g000700, Cg2g000710 and Cg2g000720) in leaves, fruit and roots of grapefruit and mandarin, using quantitative RT-PCR, in comparison to FC levels (FIGS. 7B, 7C, 7D, 7E and 8A-C). It is noted that mandarin root samples were obtained from seedlings of the polyembryonic Willowleaf, Cleopatra and Murcott mandarin varieties since they are genetically uniform, unlike seedlings of the monoembryonic Ora mandarin 31

Expression of the Cg2g000710-orthologue in Hudson grapefruit was found to be similarly high in leaves and fruitlets while ~4 fold lower in roots (FIG. 7E). In mandarin, which is homozygous for the 655 base disrupting insertion in the Cg2g000710-orthologue, expression was found to be null in all tissues: leaves, fruitlets and roots (FIGS. 7A, B and C). Expression of the Cg2g000720-orthologue was found to be high in roots in both grapefruit and mandarin, while ~135 fold lower in grapefruit fruit and leaves and essentially null in mandarin fruit and leaves. (FIGS. 7A, B and C). Expression of the Cg2g000700-orthologue was essentially null in both grapefruit and mandarin in all tissues studied. The gene expression patterns, correlated with FC levels, suggest that Cg2g000710 encodes the major C2′H directing FC biosynthesis in both leaves and fruit, while Cg2g000720 is the major C2′H directing FC biosynthesis in roots.

### Example 8

### FC Composition in Citrus Roots Differs from the Composition in Fruit and Leaves

Due to a previous report describing the identification of marmesin in trifoliate orange roots³², the present inventors examined FC/UM composition in the roots of ‘Hudson’ grapefruit, and of three polyembryonic mandarin varieties (Willowleaf, Cleopatra and Murcott), which have low to undetectable FCs/UM levels in fruit^{24,26}.

In contrast to the differential results between grapefruit vs. mandarin in fruit and leaves, in the roots the present inventors observed relatively high FC/UM levels in all of the tested varieties including both grapefruit and mandarins (FIGS. 7D and 8C). Interestingly, the composition of FCs/UM in citrus roots appears to be entirely different from those in fruit and leaf tissue; while fruit and leaves of grapefruit/pumelo accumulate mostly FCs of the “downstream” part of the biosynthesis pathway (bergamottin and 6,7-DHB, FIGS. 1, 2, 8A and B), roots accumulate high levels of demethylsuberosin (DMS) and marmesin, which are components of the “upstream” part of the FC biosynthetic pathway, just upstream of psoralen (FIGS. 1 and 8C).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Document

1. Bailey, D., Spence, J., Munoz, C. & Arnold, J. M. O. Interaction of citrus juices with felodipine and nifedipine. The Lancet 337, 268-269 (1991).
2. Bailey, D. G., Dresser, G. & Arnold, J. M. O. Grapefruit-medication interactions: Forbidden fruit or avoidable consequencesi CMAJ Can. Med. Assoc. J. 185, 309-316 (2013).
3. Bailey, D. G. Fruit juice inhibition of uptake transport: a new type of food-drug interaction: Fruit juice-drug interactions. Br. J. Clin. Pharmacol. 70, 645-655 (2010).
4. Banfield, C., Gupta, S., Marino, M., Lim, J. & Affrime, M. Grapefruit Juice Reduces the Oral Bioavailability of Fexofenadine But Not Desloratadine: Clin. Pharmacokinet. 41, 311-318 (2002).
5. Masuda, M., Watanabe, S., Tanaka, M., Tanaka, A. & Araki, H. Screening of furanocoumarin derivatives as cytochrome P450 3A4 inhibitors in citrus. J. Clin. Pharm. Ther. 43, 15-20 (2018).
6. Kane, G. C. & Lipsky, J. J. Drug-Grapefruit Juice Interactions. Mayo Clin. Proc. 75, 933-942 (2000).
7. Hanley, M. J., Cancalon, P., Widmer, W. W. & Greenblatt, D. J. The effect of grapefruit juice on drug disposition. Expert Opin. Drug Metab. Toxicol. 7, 267-286 (2011).
8. Bowers, A. G. Phytophotodermatitis. Am. J. Contact Dermat. 10, 89-93 (1999).
9. Ellis, C. R. & Elston, D. M. Psoralen-Induced Phytophotodermatitis. Dermatitis® 32, 140-143 (2021).
10. Maniam, G., Light, K. M. & Wilson, J. Margarita Burn: Recognition and Treatment of Phytophotodermatitis. J. Am. Board Fam. Med. 34, 398-401 (2021).
11. Villard, C. et al. A new P450 involved in the furanocoumarin pathway underlies a recent case of convergent evolution. New Phytol. 231, 1923-1939 (2021).
12. Karamat, F. et al. A coumarin-specific prenyltransferase catalyzes the crucial biosynthetic reaction for furanocoumarin formation in parsley. Plant J. 77, 627-638 (2014).
13. Munakata, R. et al. Molecular evolution of parsnip (Pastinaca sativa) membrane-bound prenyltransferases for linear and/or angular furanocoumarin biosynthesis. New Phytol. 211, 332-344 (2016).
14. Walling, A. L. & Walling, H. W. Phytophotodermatitis induced by wild parsnip. Dermatol. Online J. 24, (2018).
15. Vialart, G. et al. A 2-oxoglutarate-dependent dioxygenase from Ruta graveolens L. exhibits p-coumaroyl CoA 2′-hydroxylase activity (C2′H): a missing step in the synthesis of umbelliferone in plants. Plant J. 70, 460-470 (2012).
16. Alizadeh, M. et al. Recent Updates on Anti-Inflammatory and Antimicrobial Effects of Furan Natural Derivatives. J. Inflamm. Res. 13, 451-463 (2020).
17. Bourgaud, F. et al. Biosynthesis of coumarins in plants: a major pathway still to be unravelled for cytochrome P450 enzymes. Phytochem. Rev. 5, 293-308 (2006).
18. Munakata, R. et al. Parallel evolution of UbiA superfamily proteins into aromatic O-prenyltransferases in plants. Proc. Natl. Acad. Sci. 118, e2022294118 (2021).
19. Matsumoto, S., Mizutani, M., Sakata, K. & Shimizu, B.-I. Molecular cloning and functional analysis of the ortho-hydroxylases of p-coumaroyl coenzyme A/feruloyl coenzyme A involved in formation of umbelliferone and scopoletin in sweet potato, Ipomoea batatas (L.) Lam. Phytochemistry 74, 49-57 (2012).
20. Yao, R. et al. Identification and functional characterization of a p-coumaroyl CoA 2′-hydroxylase involved in the biosynthesis of coumarin skeleton from Peucedanum praeruptorum Dunn. Plant Mol. Biol. 95, 199-213 (2017).
21. Roselli, S. et al. A bacterial artificial chromosome (BAC) genomic approach reveals partial clustering of the furanocoumarin pathway genes in parsnip. Plant J. 89, 1119-1132 (2017).
22. Limones-Mendez, M. et al. Convergent evolution leading to the appearance of furanocoumarins in citrus plants. Plant Sci. 292, 110392 (2020).
23. Guo, L. & Yamazoe, Y. Inhibition of cytochrome P450 by furanocoumarins in grapefruit juice and herbal medicines. Acta Pharmacol. Sin. 25, 129-136 (2004).
24. Dugrand-Judek, A. et al. The Distribution of Coumarins and Furanocoumarins in Citrus Species Closely Matches Citrus Phylogeny and Reflects the Organization of Biosynthetic Pathways. PLoS ONE 10, e0142757 (2015).
25. Fidel, L. et al. Breeding and Analysis of Two New Grapefruit-Like Varieties with Low Furanocoumarin Content. Food Nutr. Sci. 7, 90-101 (2016).
26. Durand-Hulak, M. et al. Mapping the genetic and tissular diversity of 64 phenolic compounds in Citrus species using a UPLC-MS approach. Ann. Bot. 115, 861-877 (2015).
27. Nelson, B. K., Cai, X. & Nebenführ, A. A multicolored set of in vivo organelle markers for co-localization studies in Arabidopsis and other plants. Plant J. 51, 1126-1136 (2007).
28. Liu, Y. et al. Comparative analysis of miniature inverted-repeat transposable elements (MITEs) and long terminal repeat (LTR) retrotransposons in six Citrus species. BMC Plant Biol. 19, 140 (2019).
29. Zhang, X. & Qi, Y. The Landscape of Copia and Gypsy Retrotransposon During Maize Domestication and Improvement. Front. Plant Sci. 10, (2019).
30. Ou, S. & Jiang, N. LTR_retriever: A Highly Accurate and Sensitive Program for Identification of Long Terminal Repeat Retrotransposons. Plant Physiol. 176, 1410-1422 (2018).
31. Vardi, A., Levin, I. & Carmi, N. Induction of Seedlessness in Citrus: From Classical Techniques to Emerging Biotechnological Approaches. J. Am. Soc. Hortic. Sci. 133, 117-126 (2008).
32. Rajkumar, S. & Jebanesan, A. Bioactivity of flavonoid compounds from Poncirus trifoliata L. (Family: Rutaceae) against the dengue vector, Aedes aegypti L. (Diptera: Culicidae). Parasitol. Res. 104, 19-25 (2008).
33. Álvarez-Lugo, A. & Becerra, A. The Role of Gene Duplication in the Divergence of Enzyme Function: A Comparative Approach. Front. Genet. 12, 641817 (2021).
34. MAGADUM, S., BANERJEE, U., MURUGAN, P., GANGAPUR, D. & RAVIKESAVAN, R. Gene duplication as a major force in evolution. J. Genet. 92, 155-161 (2013).
35. Panchy, N., Lehti-Shiu, M. & Shiu, S.-H. Evolution of Gene Duplication in Plants. Plant Physiol. 171, 2294-2316 (2016).
36. Voges, M. J. E. E. E., Bai, Y., Schulze-Lefert, P. & Sattely, E. S. Plant-derived coumarins shape the composition of an Arabidopsis synthetic root microbiome. Proc. Natl. Acad. Sci. U.S.A. 116, 12558-12565 (2019).
37. Minamikawa, M. F. et al. Genome-wide association study and genomic prediction in citrus: Potential of genomics-assisted breeding for fruit quality traits. Sci. Rep. 7, 4721 (2017).
38. Yu, Y., Chen, C. & Gmitter, F. G. QTL mapping of mandarin (Citrus reticulata) fruit characters using high-throughput SNP markers. Tree Genet. Genomes 12, 77 (2016).
39. Wu, G. A. et al. Genomics of the origin and evolution of Citrus. Nature 554, 311-316 (2018).
40. Raveh, E. et al. Conventional Breeding of Cultivated Citrus Varieties. in The Citrus Genome (eds. Gentile, A., La Malfa, S. & Deng, Z.) 33-48 (Springer International Publishing, 2020). doi:10.1007/978-3-030-15308-3_4.
41. Ollitrault, P. et al. SNP mining in C. clementina BAC end sequences; transferability in the Citrus genus (Rutaceae), phylogenetic inferences and perspectives for genetic mapping. BMC Genomics 13, 13 (2012).
42. Xin, Z., Velten, J. P., Oliver, M. J. & Burke, J. J. High-Throughput DNA Extraction Method Suitable for PCR. BioTechniques 34, 820-826 (2003).
43. Bolger, A. M., Lohse, M. & Usadel, B. Trimmomatic: a flexible trimmer for Illumina sequence data. Bioinformatics 30, 2114-2120 (2014).
44. Dobin, A. et al. STAR: ultrafast universal RNA-seq aligner. Bioinformatics 29, 15-21 (2013).
45. Trapnell, C. et al. Transcript assembly and quantification by RNA-Seq reveals unannotated transcripts and isoform switching during cell differentiation. Nat. Biotechnol. 28, 511-515 (2010).
46. Wang, X.-H. et al. Identification of a diarylpentanoid-producing polyketide synthase revealing an unusual biosynthetic pathway of 2-(2-phenylethyl) chromones in agarwood. Nat. Commun. 13, 348 (2022).
47. Chang, S., Puryear, J. & Cairney, J. A simple and efficient method for isolating RNA from pine trees. Plant Mol. Biol. Report. 11, 113-116 (1993).
48. Mafra, V. et al. Reference Genes for Accurate Transcript Normalization in Citrus Genotypes under Different Experimental Conditions. PLOS ONE 7, e31263 (2012).

1. A citrus plant comprising a genome having a loss of function mutation in a Cg2g000710 gene or ortholog thereof, wherein the citrus plant is a natural producer of coumarin or furanocoumarin (FC).

2. The plant of claim 1, selected from the group consisting of pumelo (Citrus grandis), grapefruit (C. paradisi), lime (C. aurantiifolia) and sour orange (C. aurantium).

3. The plant of claim 1, selected from the group consisting of citron (C. medica) and lemon (C. limon).

4. The plant of claim 1, wherein said loss of function mutation is in both alleles of said genome.

5. The plant of claim 1, wherein said loss of function mutation is in a homozygous form.

6. The plant of claim 1, wherein said coumarin is selected from the group consisting of scopoletin and umbelliferon.

7. The plant of claim 1, wherein said FC is selected from the group consisting of bergamottin, 6′,7′-dihydroxybergamottin (6,7-DHB), marmesin, psoralen and bergaptol.

8. The plant of claim 1, wherein said loss of function mutation is selected from the group consisting of an insertion, a deletion, an insertion/deletion (indel) and a substitution.

9. The plant of claim 1, wherein said loss of function mutation is an insertion optionally, wherein said insertion is of a transposon sequence.

10. (canceled)

11. The plant of claim 9, wherein said transposon sequence is as set forth in SEQ ID NO: 37 (Copia-like LTR retrotransposon).

12. The plant of claim 1, wherein said loss of function mutation affects a C2′H and/or F6′H activity of said gene.

13. The plant part of claim 1, wherein said plant part is fruit, cutting or seed.

14. A part of the plant of claim 1, optionally wherein the plant part is a fruit.

15. (canceled)

16. A processed product of the fruit of claim 14.

17. A method of producing an edible product, comprising processing the fruit of claim 14.

18. A method of producing the plant of claim 1, the method comprising:
(a) providing a citrus plant that is a natural producer of FC;
(b) genetically modifying a Cg2g000710 gene or ortholog thereof so as to result in a loss of function mutation in the gene of ortholog thereof.

19. The method of claim 18, wherein said genetically modifying is by genome editing.

20. The method of claim 19, wherein said genetically modifying is by breeding with a citrus plant which is a non-natural producer of coumarin or FC.

21. (canceled)

22. The method of claim 18, further comprising validating loss of function of said Cg2g000710 gene or ortholog thereof.

23. The method of claim 22, wherein said validating is effected using molecular markers and/or wherein said validating is effected biochemically by testing a function of said Cg29000710 gene or ortholog thereof.

24. (canceled)
